(19) United States
(12) Patent Application Publication
(10) Pub. No.: US 2026/0060756 A1
(43) Pub. Date: Mar. 5, 2026

(54) SYSTEMS AND METHODS FOR ATTACHMENT OF A NAVIGATIONAL TRACKER

(71) Applicant: DePuy Ireland Unlimited Company, Ringaskiddy (IE)

(72) Inventors: Daniel Thommen, Oberdorf (CH); Roman Lomeli, Raynham, MA (US); Anthony Leandri, Gieres (FR); Arnaud Robert, Gieres (FR); Cory Emil, Raynham, MA (US)

(73) Assignee: DePuy Ireland Unlimited Company, Ringaskiddy (IE)

(21) Appl. No.: 19/311,695

(22) Filed: Aug. 27, 2025

Related U.S. Application Data
(60) Provisional application No. 63/687,565, filed on Aug. 27, 2024.
(60) Provisional application No. 63/687,569, filed on Aug. 27, 2024.

(51) Int. Cl. A61B 34/20; A61B 17/90; A61B 90/57

(52) U.S. Cl. CPC A61B 34/20; A61B 17/90; A61B 90/57; A61B 2034/2051

(57) ABSTRACT

A user may attach a navigational tracker to a clamping tool. The clamping tool may include a foot configured to receive a screw. For example, the foot may include a hole configured to receive the screw. The user may attach the clamping tool to a coracoid process. The user may insert a screw into the coracoid process, for example through the foot of the clamping tool. The user may remove at least a portion of the clamping tool from the coracoid process. The navigational tracker may be attached to the foot of the clamping tool. The foot of the clamping tool may remain attached to the coracoid process, for example during a surgical procedure. The navigational tracker may include at least one electromagnetic sensor.

### CROSS-REFERENCE TO PRIORITY INFORMATION

This application claims the benefit of U.S. Provisional Patent Application No. 63/687,565, filed Aug. 27, 2024, and U.S. Provisional Patent Application No. 63/687,569, filed Aug. 27, 2024, both of which are incorporated herein by reference in their entirety.

### BACKGROUND

Surgical joint repair procedures involve repair and/or replacement of a damaged or diseased joint. Many times, a surgical joint repair procedure, such as joint arthroplasty as an example, involves replacing the damaged joint with a prosthetic that is implanted into the patient's bone. Proper selection of a prosthetic that is appropriately sized and shaped and proper positioning of that prosthetic to ensure an optimal surgical outcome can be challenging. To assist with positioning, the surgical procedure often involves the use of surgical instruments to control the shaping of the surface of the damaged bone and cutting or drilling of bone to accept the prosthetic.

Virtual visualization tools are available to surgeons that use three-dimensional modeling of bone shapes to facilitate preoperative planning for joint repairs and replacements. These tools can assist surgeons with the design and/or selection of surgical guides and implants that closely match the patient's anatomy and can improve surgical outcomes by customizing a surgical plan for each patient.

### SUMMARY

A clamping tool for attachment of a navigation array to a coracoid process, the clamping tool may include an inferior leg with an inferior clamp foot located at a distal end of the inferior leg. The inferior clamp foot may be configured to contact an inferior surface of the coracoid process. The clamping tool may further include a superior leg with a superior clamp foot located at a distal end of the superior leg. The superior clamp foot may be configured to contact a superior surface of the coracoid process. The superior clamp foot may include a hole that is configured to receive a bone screw that is configured to affix the superior clamp foot to the coracoid process. Moreover, the clamping tool may include a clamp closing mechanism configured to tighten the superior clamp foot and the inferior clamp foot about the coracoid process.

The clamp closing mechanism may include a hinge. The proximate end of the inferior leg may be coupled to the hinge, and a proximate end of the superior leg is coupled to the hinge.

The clamp closing mechanism may include a tightening screw. The tightening screw may include a threaded portion and a handled portion. When the handled portion is turned clockwise, the clamp closing mechanism may be configured to decrease the distance between the inferior clamp foot and the superior clamp foot. In some examples, when the handled portion of the tightening screw is turned counterclockwise, the clamping close mechanism may be configured to increase the distance between the inferior clamp foot and the superior clamp foot. The hinge may be offset from the tightening screw. The inferior leg and the superior leg may pivot relative to each other about the hinge.

The bone screw may include a body portion and a head portion. The body portion of the bone screw may include threads and the head portion of the bone screw may be textured. The inferior clamp foot may include a first set of teeth that are configured to secure the inferior leg to the coracoid process.

The superior clamp foot may include a second set of teeth that are configured to secure the superior leg to the coracoid process. The navigational array may be attached to the distal end of the superior leg or to the closing mechanism.

The hole may define a predetermined angle through the superior clamp foot to allow for the bone screw to affix the superior clamp foot to the coracoid process.

In some examples, the inferior clamp foot may be shorter than the superior clamp foot. In some examples, the inferior clamp foot may be shifted relative to the superior clamp foot. The inferior clamp foot may also be flat or define a rounded upper surface. In some examples, the inferior clamp foot may include a foot hinge that allows for the inferior clamp foot to pivot about an inferior foot axis.

The present disclosure may include a description of a clamping tool with an elastic element, for example a preloaded spring. A clamping tool for attachment of a navigation array to a coracoid process may include an inferior leg with an inferior clamp foot located at a distal end of the inferior leg. The inferior clamp foot may be configured to contact an inferior surface of the coracoid process. The clamping tool may include a superior leg with a superior clamp foot located at a distal end of the superior leg. The superior clamp foot may be configured to contact a superior surface of the coracoid process. The superior clamp foot may include a hole that is configured to receive a bone screw that is configured to anchor the clamping tool to the coracoid process. The clamping tool may include a clamp closing mechanism with an elastic element, for example a preloaded spring, a tightening screw, and/or a hinge. The clamp closing mechanism may be configured to close the clamping tool such that the superior clamp foot and the inferior clamp foot may move closer together about the hinge.

The proximate end of the inferior leg may be coupled to the hinge, and a proximate end of the superior leg may be coupled to the hinge. The tightening screw may include a threaded portion and a handled portion. When the handled portion is turned clockwise, the clamp closing mechanism may be configured to decrease the distance between the inferior clamp foot and the superior clamp foot.

In some examples, when the handled portion of the tightening screw is turned counterclockwise, the clamping close mechanism may be configured to increase the distance between the inferior clamp foot and the superior clamp foot. The hinge may be offset from the tightening screw. The inferior leg and the superior leg may pivot relative to each other about the hingc.

In some examples, the clamping tool may open upon pressure on an approximate end of the inferior leg and an approximate end of the superior leg. The clamping tool may close upon releasing said pressure on the approximate end of the inferior leg and the approximate end of the superior leg.

In some examples, the inferior leg may include a first portion and a second portion. The first portion may end at the superior clamp foot, the second portion may end at the hinge, and the hinge may be offset from the tightening screw. An elongated axis of the tightening screw may be aligned with the first portion of the inferior leg, and the second portion may be angularly offset from the first portion.

The first end of the elastic element (e.g., preloaded spring) may be connected to the inferior leg and a second end of the elastic element (e.g., preloaded spring) may be connected to the superior leg. The first end of the clastic element (e.g., preloaded spring) may be at the opposite side of the inferior clamp foot about the hinge and the second end of the elastic element (e.g., preloaded spring) may be at the opposite side of the superior clamp foot about the hinge.

The tightening screw may be at a same side of the elastic element (e.g., preloaded spring) about the hinge, and the tightening screw may be through the inferior leg and be in contact with the superior leg. In some examples, the inferior leg may be coupled to the hinge at approximately a center of the inferior leg, and the center of the superior leg may be coupled to the hinge. The bone screw may include a body portion and a head portion. The body portion of the bone screw may include threads and the head portion of the bone screw may be textured.

The inferior clamp foot may include a first set of teeth that are configured to secure the inferior leg to the coracoid process. The superior clamp foot may include a second set of teeth that are configured to secure the superior leg to the coracoid process. The navigational array may be attached to the distal end of the superior leg or to the closing mechanism. The hole may define a predetermined angle through the superior clamp foot to allow for the bone screw to affix the superior clamp foot to the coracoid process.

In some examples, the inferior clamp foot may be shorter than the superior clamp foot. In some examples, the inferior clamp foot may be shifted relative to the superior clamp foot. The inferior clamp foot may be flat or may define a rounded upper surface. In some examples, the inferior clamp foot may include a foot hinge that allows for the inferior clamp foot to pivot about an inferior foot axis.

The present disclosure may include a description of a clamping tool with an elastic element (e.g., preloaded spring) and detachable superior clamp foot. The clamping tool for attachment of a navigation array to a coracoid process may include an inferior leg with an inferior clamp foot located at an end of the inferior leg. The inferior clamp foot may be configured to contact an inferior surface of the coracoid process. A superior leg may include an opening at an end of the superior leg, and the opening may be fitted with a detachable attachment. The clamping tool may include a clamp closing mechanism with an elastic element (e.g., preloaded spring), a tightening screw and a hinge. The clamp closing mechanism may be configured to close the clamping tool such that the superior clamp foot and the inferior clamp foot move closer together about the hinge.

The detachable attachment may be fixated to the opening through a sliding mechanism, and the detachable attachment may include two holes and a superior clamp foot. The superior clamp foot may be configured to contact a superior surface of the coracoid process. The two holes may be configured to receive bone screws that are configured to anchor the clamping tool to the coracoid process,

The two holes may define predetermined angles through the superior clamp foot to allow for the bone screw to affix the superior clamp foot to the coracoid process. The inferior leg may be coupled to the hinge at approximately a center of the inferior leg, and a center of the superior leg is coupled to the hinge.

The tightening screw may include a threaded portion and a handled portion. When the handled portion is turned clockwise, the clamp closing mechanism may be configured to decrease the distance between the inferior clamp foot and the superior clamp foot.

When the handled portion of the tightening screw is turned counterclockwise, the clamping close mechanism may be configured to increase the distance between the inferior clamp foot and the superior clamp foot. The hinge may be offset from the tightening screw. The inferior leg and the superior leg may pivot relative to each other about the hinge. The clamping tool may open upon pressure on an approximate end of the inferior leg and an approximate end of the superior leg.

The clamping tool may close upon releasing said pressure on the approximate end of the inferior leg and the approximate end of the superior leg. The inferior leg may include a first portion and a second portion. The first portion may end at the superior clamp foot, the second portion may end at the hinge, and the hinge may be offset from the tightening screw.

An elongated axis of the tightening screw may be aligned with the first portion of the inferior leg, and the second portion may be angularly offset from the first portion. A first end of the elastic element (e.g., preloaded spring) may be connected to the inferior leg and a second end of the preloaded spring may be connected to the second leg. The first end of the elastic element (e.g., preloaded spring) may be at the opposite side of the inferior clamp foot about the hinge and the second end of the clastic element (e.g., preloaded spring) may be at the opposite side of the superior clamp foot about the hinge.

The tightening screw may be at a same side of the preloaded spring about the hinge, and the tightening screw may be through the inferior leg and be in contact with the superior leg. A proximate end of the inferior leg may be coupled to the hinge, and a proximate end of the superior leg may be coupled to the hinge

The bone screw may include a body portion and a head portion. The body portion of the bone screw may include threads and the head portion of the bone screw may be textured.

The inferior clamp foot may include a first set of teeth that are configured to secure the inferior leg to the coracoid process. The superior clamp foot may include a second set of teeth that are configured to secure the superior leg to the coracoid process. The navigational array may be attached to the distal end of the superior leg or to the closing mechanism.

In some examples, the inferior clamp foot may be shorter than the superior clamp foot. The inferior clamp foot may be shifted relative to the superior clamp foot. In some examples, the inferior clamp foot may be flat or may define a rounded upper surface. In some examples, the inferior clamp foot may include a foot hinge that allows for the inferior clamp foot to pivot about an inferior foot axis.

A clamping tool may be for attachment of a navigational tracker. For example, the navigational tracker may include a (e.g., small) electro-magnetic navigational tracker. The clamping tool may be configured to attach to a coracoid process. The clamping tool may include an inferior leg including an inferior clamp foot located at a distal end of the inferior leg. The inferior clamp foot may be configured to contact an inferior surface of the coracoid process of a scapula. The clamping tool may include a superior leg including a superior clamp foot located at a distal end of the superior leg. The superior clamp foot may be configured to contact a superior surface of the coracoid process. The superior clamp foot may include an opening that may be configured to receive a navigational tracker. The clamping tool may include a closing mechanism. The closing mechanism may include a preloaded spring and/or a hinge. The closing mechanism may be configured to close the clamping tool such that the superior clamp foot and the inferior clamp foot move closer together about the hinge.

The navigational tracker may include a threaded portion configured to connect to threads of the superior clamp foot. The threads of the superior clamp foot may be disposed around the opening of the superior clamp foot, and wherein when the threaded portion of the navigational tracker may be turned clockwise, the navigational tracker may be configured to tighten the connection to the superior clamp foot. The navigational tracker may include a tip portion configured to connect to the opening of the superior clamp foot. The connection of the opening of the superior clamp foot and the tip portion of the navigational tracker may include a non-toggling connection.

The navigational tracker may include a cylindrical portion configured to connect to a clip of the superior clamp foot. The connection of the clip of the superior clamp foot and the cylindrical portion of the navigational tracker may include a non-toggling connection. The clamping tool may include an atraumatic clamping tool. The clamping tool may be configured to open upon pressure on an approximate end of the inferior leg and an approximate end of the superior leg. The clamping tool may be configured to close upon releasing said pressure on the approximate end of the inferior leg and the approximate end of the superior leg.

The closing mechanism may include a tightening screw. The tightening screw may include a threaded portion and/or a handled portion. When the handled portion is turned clockwise for example, the clamp closing mechanism may be configured to decrease a distance between the inferior clamp foot and the superior clamp foot. The hinge may be offset from the tightening screw. The inferior leg and the superior leg may be configured to pivot relative to each other about the hinge. The inferior leg may include a first portion and a second portion. The first portion may end at the superior clamp foot, the second portion may end at the hinge, and/or the hinge may be offset from the tightening screw. An elongated axis of the tightening screw may be aligned with the first portion of the inferior leg and/or the second portion may be angularly offset from the first portion.

A user may attach a clamping tool to a coracoid process of a scapula of a patient. The user may attach a navigational tracker to the clamping tool, for example after the clamping tool is attached to the coracoid process. The user may drill and/or ream a cavity, for example in a humeral bone and/or a scapula (e.g., using a surgical instrument). The surgical instrument may be a drill configured to drill and/or ream. The user may track movement of the clamping tool, for example during drilling and/or reaming of the cavity, (e.g., in the humeral bone and/or scapula) using the navigational tracker. The user may place an implant at least partially in the cavity in the humeral bone and/or scapula.

The user may drill and ream the cavity may be simultaneously. The clamping tool may include an inferior leg. The inferior leg may include an inferior clamp foot located at a distal end of the inferior leg. The inferior clamp foot may be configured to contact an inferior surface of the coracoid process. The clamping tool may include a superior leg. The superior leg may include a superior clamp foot located at a distal end of the superior leg. The superior clamp foot may be configured to contact a superior surface of the coracoid process. The superior clamp foot may include an opening that may be configured to receive the navigational tracker. The clamping tool may include a closing mechanism. The closing mechanism may include a preloaded spring and/or a hinge. The closing mechanism may be configured to close the clamping tool such that the superior clamp foot and the inferior clamp foot move closer together about the hinge.

The user may open the clamping tool by applying pressure on an approximate end of the inferior leg and an approximate end of the superior leg. The user may close the clamping tool by releasing the pressure on the approximate end of the inferior leg and the approximate end of the superior leg. The closing mechanism may include a tightening screw. The tightening screw may include a threaded portion and/or a handled portion. The user may decrease a distance between the inferior clamp foot and the superior clamp foot by turning the handled portion clockwise. The user may attach the navigational tracker to the clamping tool by threading a threaded portion of the navigational tracker to threads of the superior clamp foot. The user may attach the navigational tracker to the clamping tool by inserting a tip portion of the navigational tracker into the opening of the superior clamp foot to create a non-toggling connection. The user may attach the navigational tracker to the clamping tool by inserting cylindrical portion of the navigational tracker into the opening of the superior clamp foot to create a non-toggling connection.

A user may attach a navigational tracker to a surface of a glenoid of a scapula of a patient. The user may drill and/or ream a cavity in the glenoid using a surgical instrument (e.g., drill). The user may track movement of the glenoid, for example during drilling and/or reaming of the cavity, using the navigational tracker. The user may place an implant at least partially in the cavity in the glenoid. The user may attach the navigational tracker to the surface of the glenoid using at least one screw. The at least one screw may include a first screw and a second screw. The user may place the first screw and the second screw in a supraglenoid tubercle surface of the glenoid.

The user may place the first screw and the second screw in a supraglenoid tubercle surface of the glenoid such that the first screw and the second screw may be not parallel. The cavity in the glenoid may include a first cavity. The user may attach the navigational tracker to the surface of the glenoid by drilling a second cavity into the surface of the glenoid and/or attaching the navigational tracker to the surface of the glenoid via a pin inserted into the second cavity. The user may attach the navigational tracker to a coracoid process during the shoulder surgery. The user may drill and ream the cavity simultaneously. The user may use the navigational tracker to position a guide for the surgical instrument. The user may attach the implant to the glenoid by placing at least one screw in the glenoid using the surgical instrument.

The implant may include an ovoid implant. The user may guide the surgical instrument using the navigational tracker. The user may attach the ovoid implant to the surface of the glenoid using the surgical instrument. The implant may include a circular implant. The user may guide the surgical instrument using the navigational tracker. The user may attach the circular implant to the surface of the glenoid using the surgical instrument. The cavity in the glenoid may be comprised in a glenoid cavity of the glenoid. The user may remove the navigational tracker after the implant is placed. The user may not remove the navigational tracker during the shoulder surgery for example.

A user may attach a navigational tracker to a clamping tool. The clamping tool may include a foot configured to receive a screw. For example, the foot may include a hole configured to receive the screw. The user may attach the clamping tool to a coracoid process. The user may insert a screw into the coracoid process, for example through the foot of the clamping tool. The user may remove at least a portion of the clamping tool from the coracoid process. The navigational tracker may be attached to the foot of the clamping tool. The foot of the clamping tool may remain attached to the coracoid process, for example during a surgical procedure. The navigational tracker may include at least one electromagnetic sensor.

The user may drill and/or ream a cavity in a glenoid (e.g., glenoid cavity). The user may use the navigational tracker to position a guide for a surgical instrument. The user may use the surgical instrument to attach an implant in the cavity of the glenoid, for example by placing at least one screw in the glenoid through the implant. The user may attach the implant (e.g., in the glenoid cavity) guided by navigational assistance using the navigational tracker.

The implant may include an ovoid implant. The user may guide the surgical instrument using the navigational tracker. The user may attach the ovoid implant to the cavity of the glenoid using the surgical instrument. The implant may include a circular implant. The user may guide the surgical instrument using the navigational tracker. The user may attach the circular implant to the cavity of the glenoid using the surgical instrument.

The foot of the clamping tool may include a superior clamp foot. The clamping tool may include an inferior leg. The inferior leg may include an inferior clamp foot located at a distal end of the inferior leg. The inferior clamp foot may contact an inferior surface of the coracoid process. The clamping tool may include a superior leg, for example including the superior clamp foot. The superior clamp foot may be located at a distal end of the superior leg. The superior clamp foot may contact a superior surface of the coracoid process. The superior clamp foot may include a hole that is configured to receive the screw. The clamping tool may include a clamp closing mechanism. The clamp closing mechanism may tighten the superior clamp foot and the inferior clamp foot about the coracoid process. The user may attach a coupling interface attached to the navigational tracker to the superior clamp foot.

The user may remove the inferior leg including the inferior clamp foot and/or the clamp closing mechanism from the coracoid process. The superior clamp foot of the clamping tool and the coupling interface may remain attached to the coracoid process, for example during a surgical procedure. The user may attach the navigational tracker to the superior clamp foot, for example via the coupling interface. The user may attach the coupling interface to the superior clamp foot after the clamping tool is attached to the coracoid process.

A user may attach a navigational tracker to a clamping tool. The clamping tool may include a foot. The foot may abut a surface of a scapula. The user may attach the clamping tool to the scapula. The user may use the navigational tracker to position a guide for a surgical instrument. The user may drill and/or ream cavity in the scapula using a surgical instrument. The user may use the surgical instrument to attach an implant in the cavity of the scapula, for example by placing at least one screw in the implant. The user may attach the implant with navigation assistance using the navigational tracker.

The foot may include a first foot. The clamping tool may include the first foot and a second foot. The second foot may abut a surface of the scapula. The user may attach the first foot and the second foot to a coracoid process of the scapula. The user may drill and/or ream cavity in a glenoid of the scapula. The user may place the at least one screw in the glenoid through the implant. The user may place the first screw and a second screw in a surface of the glenoid, for example such that the first screw and the second screw are not parallel.

A user may insert a pin of a navigational tracker into a scapula. The user may use the navigational tracker to position a guide for a surgical instrument. The user may drill and ream a cavity in the scapula using a surgical instrument. The user may use the surgical instrument to attach an implant in the cavity of the scapula, for example by placing at least one screw in the implant. The user may place the implant with navigation assistance using the navigational tracker.

The scapula may include a coracoid process. The coracoid process may include a superior surface and an inferior surface. The user may insert the pin of the navigational tracker into the coracoid process laterally between the superior surface and the inferior surface of the coracoid process. The user may insert the pin of the navigational tracker into the superior surface of the coracoid process.

The scapula may include a supraglenoid tubercle. The user may insert the pin of the navigational tracker into the supraglenoid tubercle. The scapula may include a glenoid The user may insert the pin of the navigational tracker into the glenoid, for example in a surface of the glenoid proximate a supraglenoid tubercle.

### BRIEF DESCRIPTION OF THE DRAWINGS

FIG. 1 is a diagram illustrating an example of anatomical planes of a person.

FIG. 2 is an example illustrating a frontal view of a right scapula of a person.

FIG. 3A is an illustration of an example clamping tool with clamp feet and a single screw.

FIG. 3B is an example perspective view illustrating an orientation of how the clamp feet of the clamping tool of FIG. 3A can be attached to the coracoid process.

FIG. 3C is an axial view of right scapula illustrating an example orientation of how the clamping tool of FIG. 3A can be attached to the coracoid process.

FIG. 3D illustrates examples of different positions showing how a navigation array may be attached to the clamping tool of FIG. 3A while the clamping tool is applied on a coracoid process.

FIGS. 4A-4D illustrate examples of the clamping tool of FIG. 3A with alternate inferior clamp feet.

FIG. 5 illustrates an example of a clamping tool with a preloaded spring.

FIG. 6 illustrates an example procedure demonstrating how the clamping tool of FIG. 5 can be applied onto a coracoid process.

FIG. 7 illustrates a diagram that illustrates an example of how a tightening screw of the clamp closing mechanism of the clamping tool of FIG. 5 can be tightened to secure the clamping tool about the coracoid process.

FIG. 8A illustrates an example of a clamping tool with a removable superior foot.

FIG. 8B illustrates a diagram that illustrates an example of using a clamping tool with a removable superior foot in a surgical procedure for navigation assistance.

FIG. 8C illustrates an example surgical procedure with navigational assistance.

FIG. 8D illustrates another example surgical procedure with navigational assistance.

FIGS. 9A-C illustrate an example procedure demonstrating how the clamping tool of FIG. 8A may be applied onto a coracoid process.

FIGS. 9D-G illustrate example clamping tools.

FIG. 10 illustrates two different perspectives of the clamping tool of FIG. 8A.

FIG. 11 illustrates examples of different interfaces that may be used to connect a superior clamp foot to a superior leg of a clamping tool.

FIG. 12 illustrates an example of a scissor-like clamping tool.

FIG. 13 illustrates an example of a clamping tool with a rachet locking mechanism.

FIG. 14 illustrates an example of a clamping tool with a rachet locking mechanism and a preloaded spring.

FIG. 15 illustrates an example of a clamping tool with a slide fit mechanism.

FIGS. 16A-D illustrate example scapulas.

FIG. 17 illustrates example screw trajectories and drill guides.

FIG. 18 illustrates another example scapula.

FIG. 19 illustrates an example drilling procedure of a glenoid.

FIG. 20 illustrates example implants.

FIG. 21 illustrates an example of a reverse implant.

### DETAILED DESCRIPTION

There are some tools on the market for surgical procedures to fix navigation arrays on the coracoid process. However, during the surgical procedure, many tools on the market may not be properly fastened or secured as surgeons need. When navigation arrays are moved, computer programs that use the location of these navigation arrays as a reference point for surgical procedures may not generate a useful result. This particular clamping tool in the current disclosure is designed to solve the issue of rigidly fixing navigation arrays on the coracoid process so that they can be easily placed and secured to maintain the reference point.

Fixation onto the coracoid is challenging due to the high variability of form and multitude of soft tissue surrounding and attached to the bony process. The coracoacromial (CA) ligament joins the coracoid process with the acromion of the scapula, and functions in a joint stability role, primarily to prevent superior humeral head displacement. For this reason, resection of the CA ligament during primary or reverse shoulder arthroplasty may not be a standard technique. Anterolaterally, the tendon of the short head of the biceps brachii may attach to the coracoid process. Sparing this tendon may be critical to restoring mobility and function of the patient's arm following shoulder arthroplasty, because the primary biceps tendon is resected, and the muscle attachment is displaced in standard techniques. Similarly, the pectoralis minor may attach anteriorly to the coracoid, and may be spared in shoulder arthroplasty to preserve mobility and function. As a result, the superior and inferior surfaces of the coracoid process may be fixation zones for the coracoid process. The superior and inferior surfaces may be fundamentally tissue sparing because the fixation zones do not require resection of soft tissue attachments to achieve compression against bone.

This application relates to attachment of a navigation array to the scapula. The design of a bone connector for a navigation array generally needs to fulfill several, often competing requirements. One of the competing requirements may be that the connector device needs to provide adequate fixation in the bone, also for a higher variability of anatomies, so that the bone/connector interface is not moving during the procedure. On the other hand, the connector device should not be traumatic to bone and soft tissue, resp. just to an acceptable level. Another competing requirement may be that there must be a high degree of mechanical stability of the connector. On the other hand, the connector device should be slim, and should not disturb the surgical flow. A third competing requirement may be that the connector device should allow the navigation array to get as close to the tracked bone as possible, to get good navigation accuracy. On the other hand, the navigation array needs to be pushed to a certain distance, and to not visually cover the surgical site or lead to interference with other surgical instruments. The application describes solutions of example connector devices that balance these requirements for a specific surgical procedure. In some examples, the surgical procedure is a total shoulder arthroplasty, where the drilling and/or reaming step on the glenoid is navigated, to allow an accurate placement of a shoulder arthroplasty device. A variety of design solutions was found to allow a quick, stable, less traumatic way (e.g., by avoiding resection of functionally important ligaments) of attaching a navigation array to the coracoid process of the scapula.

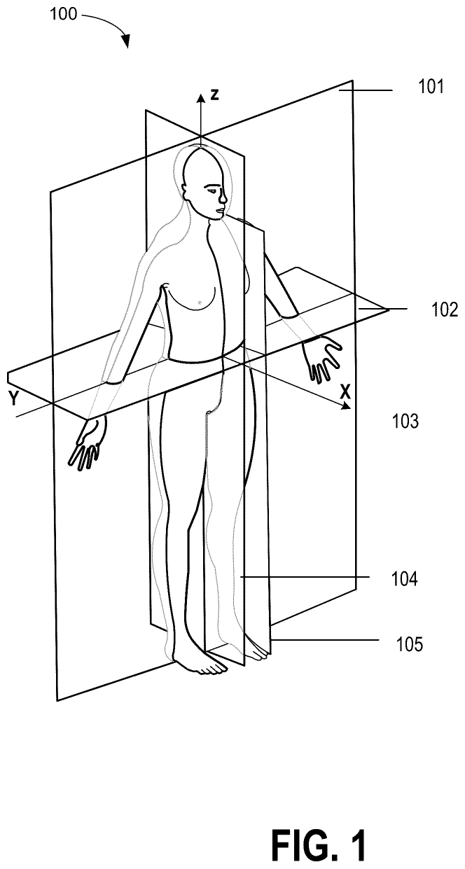

FIG. 1 is an example of anatomical planes 100. The anatomical planes 100 may be hypothetical planes used to describe the location of structures in human anatomy. The anatomical planes 100 may be applied to the human body in the anatomical position. The anatomical planes 100 may include a sagittal plane 103, a coronal plane 101 and a transverse plane 102. The sagittal plane 103 may be a vertical plane which passes through the body longitudinally. The sagittal plane 103 may be the plane on the x-z axis and may also be called longitudinal plane. The sagittal plane 103 may divide a human body into a left section and a right section. A specific sagittal plane may be the median plane 104. The median sagittal plane 104 may pass down the midline of the body, separating the body into equal halves. The parasagittal plane 105 may be a vertical cut that is off-center that separates the left of the specimen from the right in unequal portions. The parasagittal plane 105 may be parallel to the sagittal plane 103.

The coronal plane 101 may be a vertical plane that may also be called frontal plane. The coronal plane 101 may be the plane on the y-z axis. The coronal plane 101 may pass through the body longitudinally but perpendicular (at a right angle) to the sagittal plane 103. The coronal may divide the body into a front (anterior) section and back (posterior) section. The transverse plane 102 may be a horizontal plane. The transverse plane 102 may also be called horizontal plane or axis plane. The transverse plane 102 may be the plane on the x-y axis. The transverse plane 102 may be perpendicular to both the sagittal plane 103 and coronal plane 101, and parallel to the ground. The transverse plane 102 may divide the body into an upper (superior) section and a lower (inferior) section. Transverse planes may be also known as transaxial planes or axial planes.

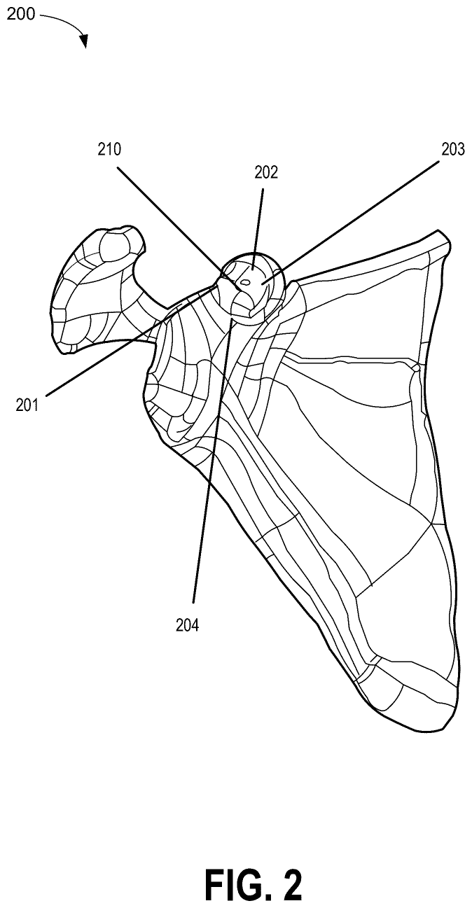

FIG. 2 is an example of a frontal view of a right scapula 200. The frontal view of right scapula 200 may also be called an Anterior Posterior (A/P) view of right scapula. The coracoid process 210 may be located at the right scapula 200. FIG. 2 shows the location of coracoid process 210 with respect to the right scapula 200. The coracoid process 210 may be a hook-shaped bone structure projecting anterolaterally from a superior aspect of a scapular neck. Surgeons may often refer to the coracoid process 210 as the “lighthouse of the shoulder” given its proximity to major neurovascular structures such as its role in guiding surgical approaches. The coracoid process 210 may also serve as a critical anchor for many tendinous and ligamentous attachments. In the present application, the example apparatuses described herein may be applied at the coracoid process during a surgical procedure. The coracoid process 210 may have multiple surfaces. For example, the coracoid process 210 may have a lateral surface 201 of the coracoid process 210, a superior surface 202 of the coracoid process 210, a medial surface 203 of the coracoid process 210, and an inferior surface 204 of the coracoid process 210. Different apparatus may be applied on different surfaces of the coracoid process to facilitate a surgical procedure.

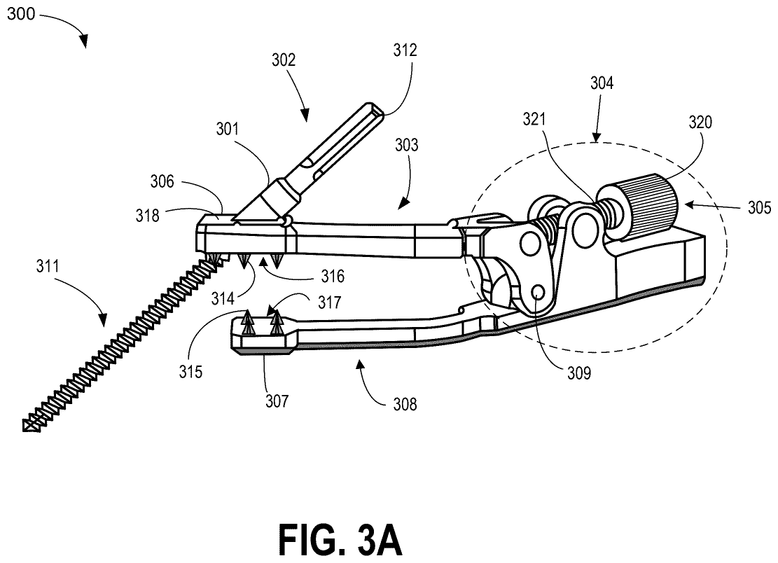

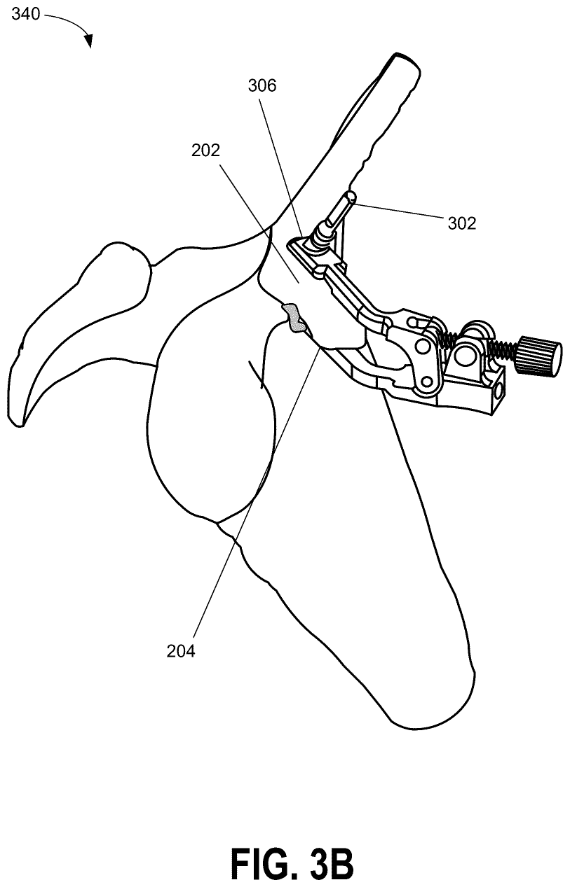

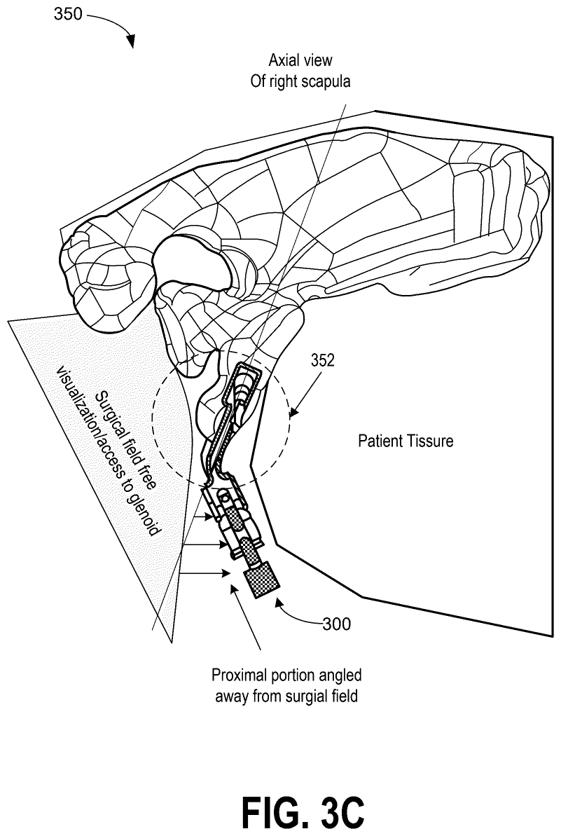

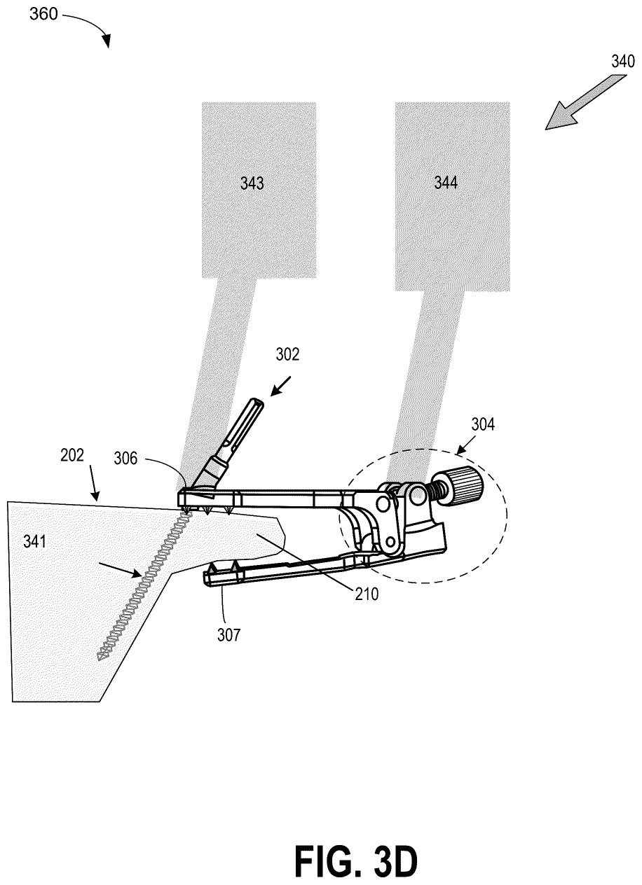

FIG. 3A is an example of a clamping tool 300. FIGS. 3B, 3C and 3D illustrate perspective views of the clamping tool 300 with a navigation array attached to the clamping tool 300 and the clamping tool attached to a coracoid process (e.g., the coracoid process 210). The clamping tool 300 may be applied on the coracoid process for a surgical procedure. The clamping tool 300 may include a superior leg 303 and an inferior leg 308. The superior leg 303 and the inferior leg 308 may be fixed via a clamp closing mechanism 304 of the clamping tool 300.

The superior leg 303 may extend from a proximal end, which is coupled to the clamp closing mechanism 304, to a distal end. The superior leg 303 may include a superior clamp foot 306 that is located at the distal end of the superior leg 303 with respect to the closing mechanism 304. An inner surface 316 of the superior clamp foot 306 may be configured to contact a superior surface of the coracoid process (e.g., the superior surface 202 of the coracoid process 210) when the clamping tool 300 is being used during a surgical procedure. For example, the superior clamp foot 306 may include clamp teeth 314 that extend from the inner surface 316 of the superior clamp foot 306. As shown in FIG. 3A, in some examples, the superior clamp foot 306 may include a plurality of clamp teeth 314 (e.g., six clamp teeth) that are configured to engage the superior surface of the coracoid process to secure the superior leg 303 to the coracoid process. As described in more detail herein, in other examples, the superior clamp foot 306 may take different shapes and/or include a different number of clamp teeth 314.

The inferior leg 308 may extend from a proximal end, which is coupled to the clamp closing mechanism 304, to a distal end. The inferior leg 308 may include an inferior clamp foot 307 at the distal end of the inferior leg 308 with respect to the closing mechanism 304. An inner surface 317 of the inferior clamp foot 307 may be configured to contact an inferior surface of a coracoid process (e.g., the inferior surface 204 of the coracoid process 210) when the clamping tool 300 is being used during a surgical procedure. For example, the inferior clamp foot 307 may include clamp teeth 315 that extend from the inner surface 317 of the inferior clamp foot 307. For example, the inferior clamp foot 307 may include a plurality of clamp teeth 315 (e.g., four clamp teeth) that are configured to engage the inferior surface of the coracoid process to secure the inferior leg 308 to the coracoid process. As described in more detail herein, in other examples, the inferior clamp foot 307 may take different shapes and/or include a different number of clamp teeth 315.

In some examples, the superior leg 303 and/or the inferior leg 308 may be bent or curved (e.g., as shown in FIG. 3C). In other examples, the superior leg 303 and/or the inferior leg 308 may be relatively straight. Further, in some examples, the superior leg 303 may be longer than the inferior leg 308. For example, the distance between an end of the superior clamp foot 306 and the closing mechanism 304 may be greater than an end of the inferior clamp foot 307 and the closing mechanism 304.

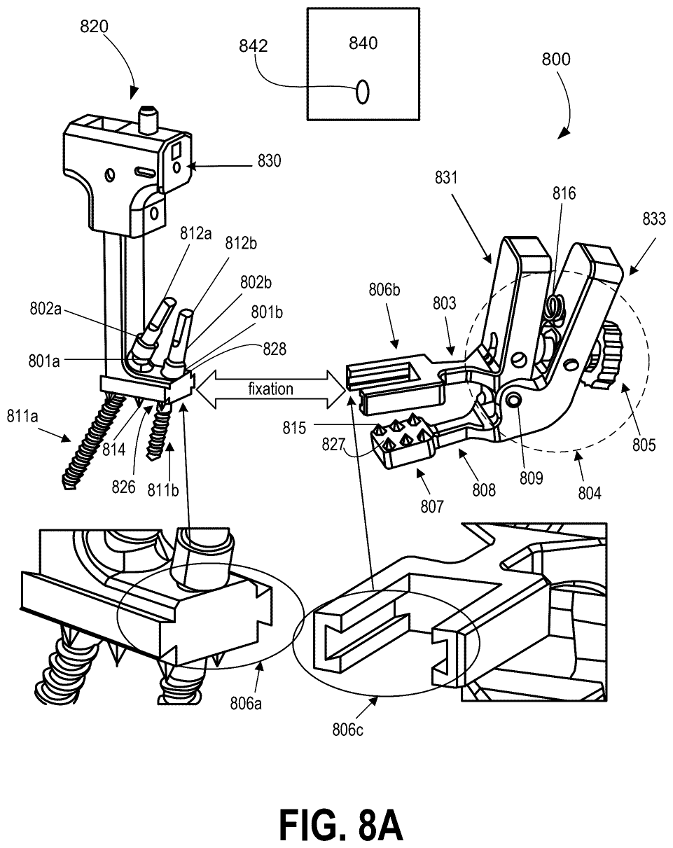

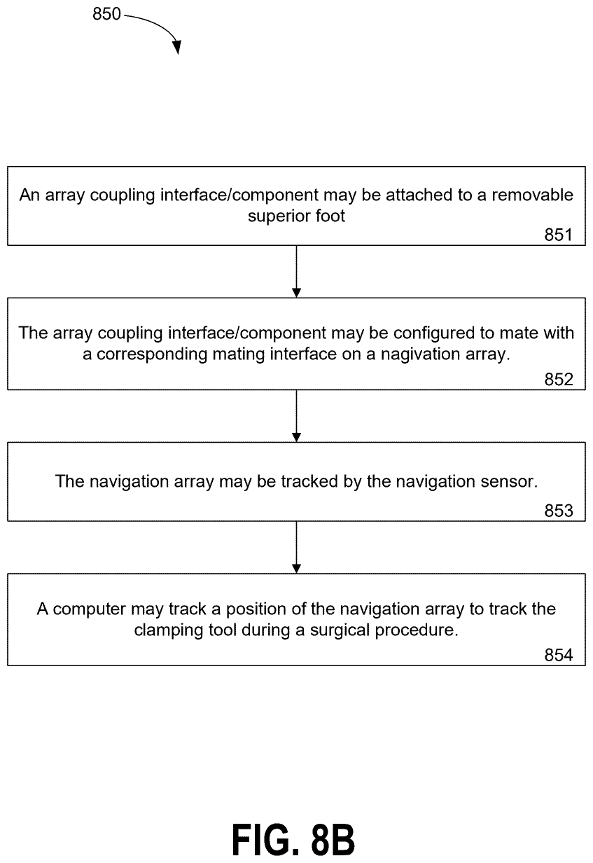

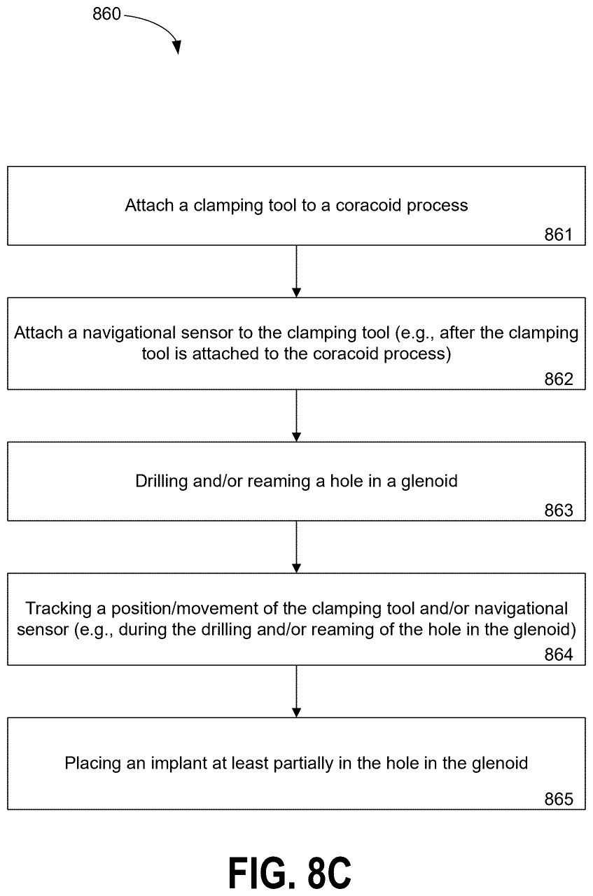

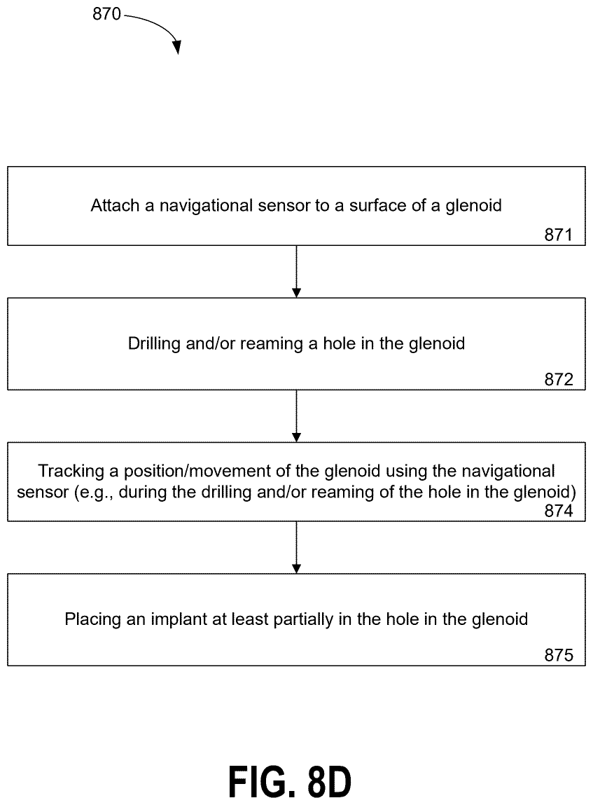

The superior clamp foot 306 may be integral with the superior leg 303 and the inferior clamp foot 307 may be integral with the inferior leg 308. In other examples, the superior clamp foot 306 may be a detachable part that is fixated to the superior leg 303 (e.g., as described with reference to FIGS. 8-11). Further, although not illustrated, in some examples, the inferior clamp foot 307 may be a detachable part that is fixated to the inferior leg 308.

The superior clamp foot 306 may include one or more holes, such as a hole 301. The hole 301 may be configured to receive a bone screw 302. The bone screw 302 may be configured to affix the superior clamp foot 306 to the coracoid process. In some examples, the hole 301 in the superior clamp foot 306 may be at an angle with respective to the superior clamp foot 306. For example, the hole 301 may pass through the superior clamp foot 306 at an angle to allow the bone screw 302 to pass from an outer surface 318 of the superior clamp foot 306 to the inner surface 316 of the superior clamp foot 306 to secure the superior clamp foot 306 to the superior surface of the coracoid process.

The bone screw 302 may include a head 312 and a body 311. In some examples, the body 311 of the bone screw 302 may contain threads, and the hole 301 may contain corresponding internal threads to facilitate the receipt of the bone screw 302. The thread of the hole 301 and the thread of the bone screw 302 may be used to tighten a fixation between the superior clamp foot 306 and the bone screw 302. When the superior clamp foot 306 is fixated to the bone screw 302, the head 312 of the bone screw may be exposed at the outer surface 318 of the superior clamp foot 306 and the body 311 of the bone screw may be exposed through the inner surface 316 of the superior clamp foot 306. The head 312 of the bone screw 302 may be textured (e.g., to improve a grip on the bone screw).

As noted above, the clamping tool 300 may include a closing mechanism 304 that is coupled at a proximate end of the superior leg 303 and at a proximate end of the inferior leg 308. The closing mechanism 304 may include a hinge 309 and a tightening screw 305. The hinge 309 may be offset from the tightening screw 305. The hinge 309 may be made of various materials, such as metal, plastic, or steel. The hinge 309 may connect to the proximate ends of the superior leg 303 and the inferior leg 308. As described in more detail herein, the superior leg 303 and the inferior leg 308 may pivot relative to each other about the hinge 309. As such, the hinge 309 may provide a degree of freedom between the movement of the superior leg 303 and inferior leg 308. As described in more detail herein, when the superior leg 303 and the inferior leg 308 move with respect to each other about the hinge 309, the distance between the superior clamp foot 306 and the inferior clamp foot 307 may become larger or smaller.

The tightening screw 305 may include a head 320 and a threaded body portion 321. The threaded body portion 321 of tightening screw 305 may contain threads. A proximate end of the threaded body portion 321 may terminate at the head 320 of the tightening screw 305, while a distal end (e.g., point) of the threaded body portion 321 may be configured to contact the superior leg 303. For example, the distal end, or point, of the threaded body portion 321 of the tightening screw 305 may be secured to the proximate end of the superior leg 303, such that the head 320 of the tightening screw 305 moves toward the hinge 309 when rotated clockwise. As described in more detail herein, as the head 320 of the tightening screw 305 is rotated clockwise, the point of the threaded body portion 321 may cause the superior leg 303 to pivot about the hinge 309 to cause the superior clamp foot 306 to move closer to the inferior clamp foot 307 (e.g., cause the superior and inferior clamp feet to close). As the head 320 of the tightening screw 305 is rotated counterclockwise, the point of the threaded body portion 321 may cause the superior leg 303 to pivot about the hinge 309 to cause the superior clamp foot 306 to move away from to the inferior clamp foot 307 (e.g., cause the superior and inferior clamp fect to open).

The threaded body portion 321 of the tightening screw 305 may be in contact with the superior leg 303. The head 320 of the tightening screw 305 may be textured (e.g., to improve a grip on the tightening screw).

As noted above, as the head 320 of the tightening screw 305 is rotated clockwise, the point of the tightening screw 305 may cause the superior leg 303 to pivot about the hinge 309 to cause the superior clamp foot 306 to move closer to the inferior clamp foot 307. For instance, as the head 320 of the tightening screw 305 is rotated clockwise, the tightening screw 305 may push the superior leg 303 forwards to cause the superior leg 303 to pivot about the hinge 309 to cause the superior clamp foot 306 to move towards the inferior clamp foot 307. This clockwise rotation of the head 320 may be performed to tighten the superior and inferior clamp feet about the coracoid process. The tightening screw 305 may restrain movement of superior leg 303 and inferior leg 308 in order to maintain pressure of surfaces superior clamp foot 306 and inferior clamp foot 307 upon the coracoid process.

As the head 320 of the tightening screw 305 is rotated counterclockwise, the point of the threaded body portion 321 of the tightening screw 305 may cause the superior leg 303 to pivot about the hinge 309 to cause the superior clamp foot 306 to move away from the inferior clamp foot 307. For instance, as the head 320 of the tightening screw 305 is rotated counterclockwise, the tightening screw 305 may pull the superior leg 303 backwards to cause the superior leg 303 to pivot about the hinge 309 to cause the superior clamp foot 306 to move away from the inferior clamp foot 307. This counterclockwise rotation of the head 320 may be performed to loosen the superior and inferior clamp feet and allow the clamping tool 300 may be removed from the coracoid process.

FIGS. 3B, 3C and 3D illustrate example systems that include a navigation array attached to the clamping tool 300 while the clamping tool 300 is applied on a coracoid process (e.g., the coracoid process 210). When the clamping tool 300 is clamped to the coracoid process, the clamp teeth 315 may be in contact with the coracoid process. Tracking the coracoid process may be crucial in areas related to a shoulder surgical procedure. Accurate positioning of surgical equipment may have significant implications during a shoulder surgical procedure. The location of the coracoid process may serve as a reference point during a shoulder surgical procedure. Being able to accurately track the coracoid process may provide great precision in procedures like implant placement, especially on the glenoid. The navigation array may be able to track the location of the coracoid process. In the present application, the clamping tool 300 may ensure the navigation array is properly fixed onto the coracoid process and restrict the movement of the navigation array. In this way, computer programs that use the location of these navigation arrays as a reference point for surgical procedures may generate useful results.

FIG. 3B is a first perspective view illustrating an orientation of how the clamp feet are attached to the coracoid process. At 340, the superior clamp foot 306 is contacting the superior surface 202 of the coracoid process 210. The inferior clamp foot 307 is contacting the inferior surface 204 of the coracoid process 210. This way of fixing the clamping tool onto the coracoid process is less traumatic compared to the traditional fixing of a clamping tool by contacting the medial and lateral surface of the coracoid process.

In one example, a first end of the bone screw 302 may be embedded in bone (e.g., either in hole separately drilled or may be impacted into unprepared bone) through the superior clamp foot 306 of the clamping tool 300.

FIG. 3C is an axial view of right scapula illustrating an orientation of how the clamping tool 300 is attached to the coracoid process. In the diagram 350, the proximal extension 352 of the clamping tool 300 may be angled away, either medially, laterally, or superiorly, relative to the longitudinal direction of the coracoid process. This angled away design may allow the proximal extension 352, where the navigation array is attached, to be guided along a periphery of the surgical field. This angled away design may prevent visual coverage of the surgical field, of interference with the surgical instruments or the practitioner.

As shown in FIG. 3C, the proximal portion of clamping tool 300 may be angled away from surgical field. As a result, a practitioner may have surgical field free visualization or surgical free access to a glenoid. A practitioner may also have an unobstructed view of patient tissue on the right side of the clamping tool 300 as shown in FIG. 3C.

FIG. 3D illustrates examples of different positions a navigation array may be attached to a clamping tool applied on a coracoid process. In the diagram 360, the coracoid process base 341 or the body of the bone screw 302 may be embedded in the bone (e.g., in hole separately drilled or impacted into an unprepared bone) through the superior clamp foot 306 of the clamping tool 300. The bone screw 302 may be positioned through the superior clamp foot 306.

In an example, a tracking system may be used to track the relative position and orientation of surgical instruments and patient anatomical structure(s) for surgical navigation. The tracking system may include a navigation sensor (not shown) (e.g., optical sensor/camera, electromagnetic sensor, etc.) for determining the position and orientation of one or more navigation arrays 343 or 344 that include one or more markers (not shown), for example, optical markers, electromagnetic markers, or other types of markers. The navigation arrays 343, 344 may be coupled to the clamping tool 300, which may be secured to a patient's anatomical structure so that the navigation sensor may track the position and orientation of the patient's anatomy. The clamping tool 300 may be configured to secure one or more navigation arrays, for example at the superior clamp foot 306 and/or at the closing mechanism 304.

Data may be collected using the navigation arrays 343, 344 and the navigation sensor and processed using a processing device with a memory or a storage device to register the patient anatomy to corresponding points in pre-operative imaging or models associated with a surgical plan. Further, intraoperative data may be collected using the navigation arrays 343, 344 and the navigation sensor to track the relative position and orientation of surgical instruments and patient anatomical structure(s) to provide surgical navigation in accordance with a surgical plan.

In a surgical procedure, tracking may be initiated continuously and automatically. During a surgical procedure many events may occur (e.g., patient movement, instrument movement, loss of tracking, etc.) that may disturb the tracking process. A computer (e.g., comprising one or more computer programs) may be implemented to verify and adjust tracking parameters continuously or periodically. The computer may continuously track a position of the navigation arrays 343 or 344 and utilize the tracked position to guide surgical steps.

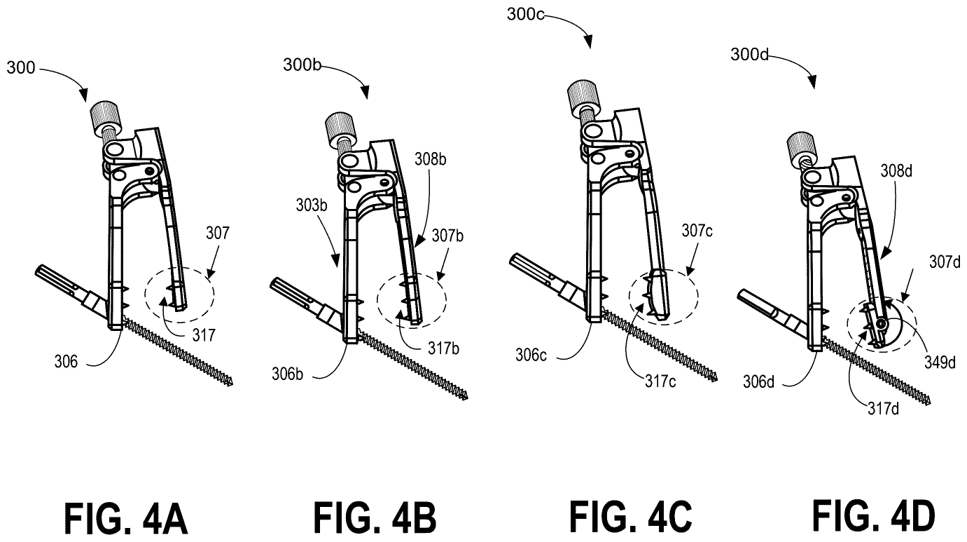

The inferior clamp foot of the clamping tool 300 may have different shapes, sizes and surface geometries to interact with the coracoid process. For example, FIGS. 4A-4D illustrate examples of different versions of an inferior clamp foot, such as the inferior clamp foot 307 of the clamping tool 300.

FIG. 4A illustrates an example of a short inferior clamp foot 307. The inferior clamp foot 307 may be shorter than the superior clamp foot 306 of the clamping tool 300. For example, the inferior clamp foot 307 may have less clamp teeth than the superior clamp foot 306. In one embodiment, the superior clamp foot 306 may have three sets of clamp teeth and the inferior clamp foot 307 may have two sets of clamp teeth. The inferior clamp foot 307 may have clamp teeth on an inner surface 317 of the inferior clamp foot 307, and the inner surface 317 of the inferior clamp foot 307 may be flat. When the clamping tool 300 closes about the closing mechanism 304, the superior clamp foot 306 and the inferior clamp foot 307 may extend similar length from the closing mechanism.

FIG. 4B illustrates an example of a shifted inferior clamp foot 307b. The inferior clamp foot 307b of a clamping tool 300b may be shifted relative to the superior clamp foot 306b. For example, the inferior clamp foot 307b may be of similar size as the superior clamp foot 306b. When the superior leg 303b and the inferior leg 308b close about the closing mechanism, the superior clamp foot 306b and the inferior clamp foot 307b may be shifted. The superior leg 303b may extend further from the closing mechanism than the inferior leg 308b. The superior clamp foot 306b may also extend further from the closing mechanism than the inferior clamp foot 307b. The inferior clamp foot 307b may have clamp teeth on an inner surface 317b of the inferior clamp foot 307b, and the inner surface 317b of the inferior clamp foot 307b may be flat. In one embodiment, the superior clamp foot 306b may have three sets of clamp teeth and the inferior clamp foot 307b may have three sets of clamp teeth.

FIG. 4C illustrates an example of a rounded inferior clamp foot 307c. The inferior clamp foot 307c of a clamping tool 300c may be rounded. For example, unlike the clamping tool 300 where the inner surface 317 of the inferior clamp foot 307 may be flat, an inner surface 317c of the inferior clamp foot 307c may be rounded. An opposite surface of the inferior clamp foot 307c may be flat. The clamp teeth of the inferior clamp foot 307c may be located on the round surface 317c of the inferior clamp foot 307c. When the clamping tool 300c closes about the closing mechanism, the superior clamp foot 306c and the inferior clamp foot 307c may extend similar length from the closing mechanism. In one embodiment, the superior clamp foot 306c may have three sets of clamp teeth and the inferior clamp foot 307c may have three sets of clamp teeth.

FIG. 4D illustrates an example of a hinged inferior clamp foot 307d. The inferior clamp foot 307d of a clamping tool 300d may be attached to an inferior leg 308d via a hinge 349d. The inferior clamp foot 307d may be able to rotate (e.g., pivot) about the hinge 349d on the inferior leg 308d with a degree of freedom. The inferior clamp foot 407d may have clamp teeth on an inner surface 317d of the inferior clamp foot, and the inner surface 317d of the inferior clamp foot may be flat. When the clamping tool 300d closes about the closing mechanism, the superior clamp foot 306d and the inferior clamp foot 307d may extend similar length from the closing mechanism. In one embodiment, the superior clamp foot 306d may have three sets of clamp teeth and the inferior clamp foot 307d may have three sets of clamp teeth.

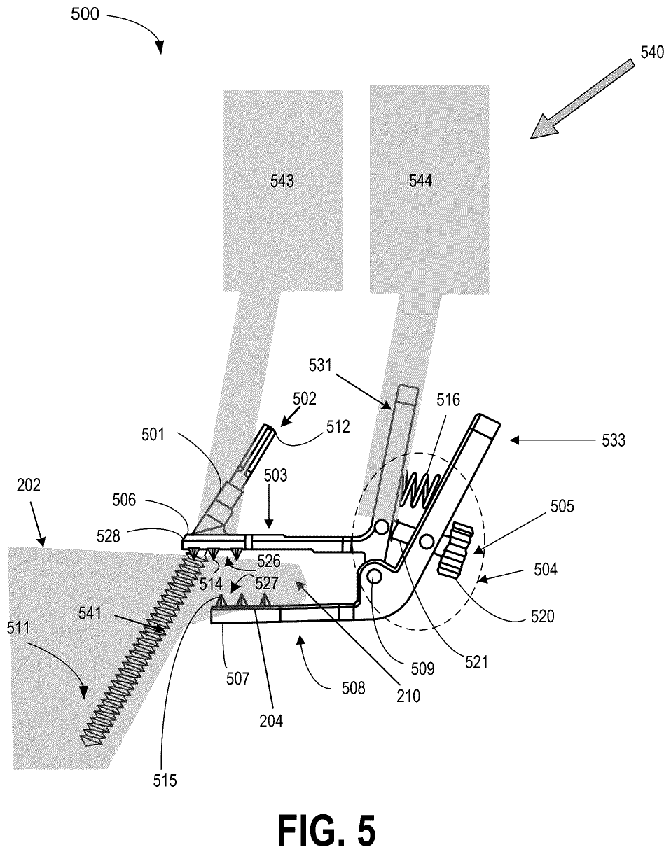

FIG. 5 illustrates an example of a preloaded clamp 500. The preloaded clamp 500 is a clamping tool with a preloaded spring. The preloaded clamp 500 may include a superior leg 503 and an inferior leg 508. The superior leg 503 and the inferior leg 508 may be fixed via a clamp closing mechanism 504 of the clamping tool 500.

The superior leg 503 may extend from a proximal end, which is coupled to the clamp closing mechanism 504, to a distal end. A superior handle 531 may be connected to the superior leg 503 at the closing mechanism 504. The superior leg 503 may include a superior clamp foot 506 that is located at the distal end of the superior leg 503 with respect to the closing mechanism 504. An inner surface 526 of the superior clamp foot 506 may be configured to contact a superior surface of the coracoid process (e.g., the superior surface 202 of the coracoid process 210) when the clamping tool 500 is being used during a surgical procedure. For example, the superior clamp foot 506 may include clamp teeth 515 that extend from the inner surface 526 of the superior clamp foot 506. In some examples, the superior clamp foot 506 may include a plurality of clamp teeth 514 (e.g., six clamp teeth) that are configured to engage the superior surface of the coracoid process to secure the superior leg 503 to the coracoid process. In other examples, the superior clamp foot 506 may take different shapes and/or include a different number of clamp teeth 514.

The inferior leg 508 may extend from a proximal end, which is coupled to the clamp closing mechanism 504, to a distal end. An inferior handle 533 may be connected to the inferior leg 508 at the closing mechanism 504. The inferior leg 508 may include an inferior clamp foot 507 at the distal end of the inferior leg 508 with respect to the closing mechanism 504. An inner surface 527 of the inferior clamping foot 507 may be configured to contact an inferior surface of a coracoid process (e.g., the inferior surface 204 of the coracoid process 210) when the clamping tool 500 is being used during a surgical procedure. For example, the inferior clamp foot 507 may include clamp teeth 515 that extend from the inner surface 527 of the inferior clamping foot 507. For example, the inferior clamp foot 507 may include a plurality of clamp teeth 515 (e.g., four clamp teeth) that are configured to engage the inferior surface of the coracoid process to secure the inferior leg 508 to the coracoid process. In other examples, the inferior clamp foot 507 may take different shapes and/or include a different number of clamp teeth 515 as described in FIG. 4A-4D.

In some examples, the superior leg 503 and/or the inferior leg 508 may be bent or curved. In other examples, the superior leg 503 and/or the inferior leg 508 may be relatively straight. Further, in some examples, the superior leg 503 may be longer than the inferior leg 508. For example, the distance between an end of the superior clamp foot 506 and the closing mechanism 504 may be greater than an end of the inferior clamp foot 507 and the closing mechanism 504.

The superior clamp foot 506 may be integral with the superior leg 503 and the inferior clamping foot 507 may be integral with the inferior leg 508. In other examples, the superior clamp foot 506 may be a detachable part that is fixated to the superior leg 503. Further, although not illustrated, in some examples, the inferior clamping foot may be a detachable part that is fixated to the inferior leg.

The superior clamp foot 506 may include one or more holes, such as a hole 501. The hole 501 may be configured to receive a bone screw 502. The bone screw 502 may be configured to affix the superior clamp foot 506 to the coracoid process. In some examples, the hole 501 in the superior clamp foot 506 may be at an angle with respective to the superior clamp foot 506. For example, the hole 501 may pass through the superior clamp foot 506 at an angle to allow the bone screw 502 to pass from an outer surface 528 of the superior clamp foot 506 to the inner surface 526 of the superior clamp foot 506 to secure the superior clamp foot 506 to the superior surface of the coracoid process.

The bone screw 502 may include a head 512 and a body 511. In some examples, the body 511 of the bone screw 502 may contain threads, and the hole 501 may contain internal threads to facilitate the receipt of the bone screw 502.

The thread of the hole 501 and the thread of the bone screw 502 may be used to tighten a fixation between the superior clamp foot 506 and the bone screw 502. When the superior clamp foot 506 is fixated to the bone screw 502, the head 512 of the bone screw may be exposed at the outer surface 528 of the superior clamp foot 506 and the body 511 of the bone screw may be exposed through the inner surface 526 of the superior clamp foot 506. The head 512 of the bone screw 502 may be textured (e.g., to improve a grip on the bone screw).

As noted above, the preloaded clamp 500 may include a closing mechanism 504 that is coupled at a proximate end of the superior leg 503 and at a proximate end of the inferior leg 508. The closing mechanism 504 may include a hinge 509, a preloaded spring 516, and a tightening screw 505. An elongated axis of the tightening screw 505 may be aligned with a first portion of the inferior leg 503. A second portion of the inferior leg 503 may be angularly offset from the first portion of the inferior leg 503. The hinge 509 may be offset from the tightening screw 505. The hinge 509 may be made of various materials, such as metal, plastic, or steel. The hinge 509 may connect to proximate ends of the superior leg 503 and the inferior leg 508. As described in more detail herein, the superior leg 503 and the inferior leg 508 may pivot relative to each other about the hinge 509. As such the hinge 509 may provide a degree of freedom between the movement of the superior leg 503 and inferior leg 508. As described in more detail herein, when the superior leg 503 and the inferior leg 508 move with respect to each other about the hinge 509, the distance between the superior clamp foot 506 and inferior clamp foot 507 may become larger or smaller.

The preloaded clamp 500 may include a preloaded spring 516. One end of the preloaded spring 516 may be connected to the superior handle 531 and the other end of the preloaded spring 516 may be connected to the inferior handle 533. The superior handle 531 may be located on an opposite side of the hinge 509 with respect to the superior clamp foot 506. The inferior handle 533 may be located on an opposite side of the hinge 509 with respect to the inferior clamp foot 507.

The preloaded clamp 500 may allow a quick and simple way of attachment. At a preload state, the preloaded spring 516 may be preloaded with potential energy. The preloaded spring 516 may push the superior handle 531 and the inferior handle 533 apart to cause the superior clamp foot 506 and the inferior clamp foot 507 to be closer to each other. When the superior handle 531 and the inferior handle 533 are pressed together or being squeezed, the preloaded spring 516 may be squeezed and compressed, and thus the potential energy of the preloaded spring 516 may increase. As the pressure applied on the superior handle 531 and the inferior handle 533 increases, the distance between the superior handle 531 and inferior handle 533 may decrease. The increased pressure between the superior handle 531 and inferior handle 533 may cause the clamping tool to open. In other words, the increased pressure may cause the distance between the superior clamp foot 506 and the inferior clamp foot 507 to increase because the superior handle 531 is connected to the superior clamp foot 506 and the inferior handle 533 is connected to the inferior clamp foot 507. The open preloaded clamp 500 with pressure on the superior handle 531 and the inferior handle 533 may be brought over to a bone, for example the coracoid process. Once a desired location to secure the preloaded clamp 500 is confirmed, the pressure on the superior handle 531 and the inferior handle 533 may then be released. The compressed preloaded spring 516 may have potential energy. As the pressure is released, the preloaded spring 516 may expand and exert pressure on the superior handle 531 and the inferior handle 533 causing the distance between the superior handle 531 and the inferior handle 533 to increase. The distance between the superior clamp foot 506 and the inferior clamp foot 507 may decrease because the superior handle 531 is connected to the superior clamp foot 506 and the inferior handle 533 is connected to the inferior clamp foot 507. The superior clamp foot 506 and the inferior clamp foot 507 may become in contact with the coracoid process and squeeze the coracoid process because the preloaded spring 516 expands. The superior clamp foot 506 and the inferior clamp foot 507 may clamp onto the coracoid process and the preloaded clamp 500 may be held in place by the preloaded spring 516.

The preloaded clamp 500 may include the tightening screw 505 to further restrict the movement of the preloaded clamp 500. The tightening screw 505 may include a head 520 and a body 521 portion. A proximate end of the body portion may terminate at the head of the tightening screw, while a distal end (e.g., point) of the body portion may be configured to contact the superior handle 531. For example, the distal end, or point, of the body portion of the tightening screw 505 may be secured to the superior handle 531, such that the head 520 of the tightening screw 505 moves toward the hinge 509 when rotated clockwise. As described herein, as the head 520 of the tightening screw 505 is rotated clockwise, the point of the body portion may cause the superior handle 531 to pivot about the hinge 509 to cause the superior clamp foot 506 to move closer to the inferior clamp foot 507 (e.g., cause the superior and inferior clamp feet to close). As the head of the tightening screw 505 is rotated counterclockwise, the point of the body portion may cause the superior handle 531 to pivot about the hinge 509 to cause the superior clamp foot 506 to move away from to the inferior clamp foot 507 (e.g., cause the superior and inferior clamp fect to open).

When the tightening screw 505 is positioned through the inferior handle 533, the body 521 portion of the tightening screw 505 may be in contact with the superior handle 531, and the head 520 of the tightening screw 505 may be exposed at the opposite side of the inferior handle 533. The head 520 of the tightening screw 505 may be textured (e.g., to improve a grip on the tightening screw).

As noted above, as the head of the tightening screw 505 is rotated clockwise, the point of the tightening screw 505 may cause the superior handle 531 to pivot about the hinge 509 to cause the superior clamp foot 506 to move closer to the inferior clamp foot 507. For instance, as the head of the tightening screw 505 is rotated clockwise, the tightening screw 505 may push the superior handle 531 forwards to cause the superior leg 503 to pivot about the hinge 509 to cause the superior clamping foot 506 to move towards the inferior clamping foot 507. This clockwise rotation of the head 512 may be performed to tighten the superior clamp foot 506 and the inferior clamping foot 507 about the coracoid process. The tightening screw 505 may restrain movement of the superior leg 503 and the inferior leg 508 in order to maintain pressure of surfaces of the superior clamp foot 506 and the inferior clamp foot 507 upon the coracoid process. Once adequate resistance is felt, the rotation of the tightening screw 505 may be stopped. The tightening screw 505 may then be pulled outwards away from the inferior handle 533 to cause the tightening screw 505 to lock in place. Once tightening screw 505 is locked in place, the movement of the tightening screw 505 may be restricted and may not be further rotated. The bone screw 502 may then be inserted into the coracoid process through the hole 501 to further restrict the movement of the preloaded clamp 500. The bone screw 502 may provide the reloaded clamp 500 additional anchorage onto the coracoid process.

As the head 512 of the tightening screw 505 is rotated counterclockwise, the point of the body portion of the tightening screw 505 may cause the superior leg 503 to pivot about the hinge 509 to cause the superior clamping foot 506 to move away from the inferior clamping foot 507. For instance, as the head 512 of the tightening screw 505 is rotated counterclockwise, the tightening screw 505 may pull the superior leg 503 backwards to cause the superior leg 503 to pivot about the hinge 509 to cause the superior clamping foot 506 to move away from the inferior clamping foot 507. This counterclockwise rotation of the head may be performed to loosen the superior and inferior clamping feet and allow the preloaded clamp 500 to be removed from the coracoid process.

Coracoid process base 541 or the body of the bone screw 511 may be embedded in bone (e.g., either in hole separately drilled or may be impacted into unprepared bone) through the superior clamp foot 506 of the preloaded clamp 500. A surgical procedure may use the navigation array that is attached to the camping tool for surgical navigation. The navigation array may be a tracking device that is attached to the preloaded clamp 500, and the tracking device may be then tracked by a navigation camera. The navigation array and the navigation camera may be used throughout the surgical procedure. The bone screw 502 may be positioned through the superior clamp foot 506.

In an example, a tracking system may be used to track the relative position of surgical instrument(s) and patient anatomical structure(s) for surgical navigation. The tracking system may include a navigation sensor (not shown) (e.g., optical sensor/camera, electromagnetic sensor, etc.) for determining the position and orientation of one or more navigation arrays 543 or 544 that include one or more markers (not shown), for example, optical markers, electromagnetic markers, or other types of markers. The navigation arrays 543, 544 may be coupled to the preloaded clamp 500, which may be secured to a patient's anatomical structure so that the navigation sensor may track the position and orientation of the patient's anatomy. The preloaded clamp 500 may be configured to secure one or more navigation arrays 543, 544, for example at the superior clamp foot 506 and/or at the closing mechanism 504.

Data may be collected using the navigation arrays 543, 544 and navigation sensor and processed using a processing device with a memory or a storage device to register the patient anatomy to corresponding points in pre-operative imaging or models associated with a surgical plan. Further, intraoperative data may be collected using the navigation arrays 543, 544 and navigation sensor to track the relative position and orientation of surgical instruments and patient anatomical structure(s) to provide surgical navigation in accordance with a surgical plan.

In a surgical procedure, tracking may be initiated continuously and automatically. During a surgical procedure, many events may occur (e.g., patient movement, instrument movement, loss of tracking, etc.) that may disturb the tracking process. A computer may be implemented to verify and adjust tracking parameters continuously or periodically. The computer may continuously track a position of the navigation array 543 or 544 and utilize the tracked position to guide surgical steps.

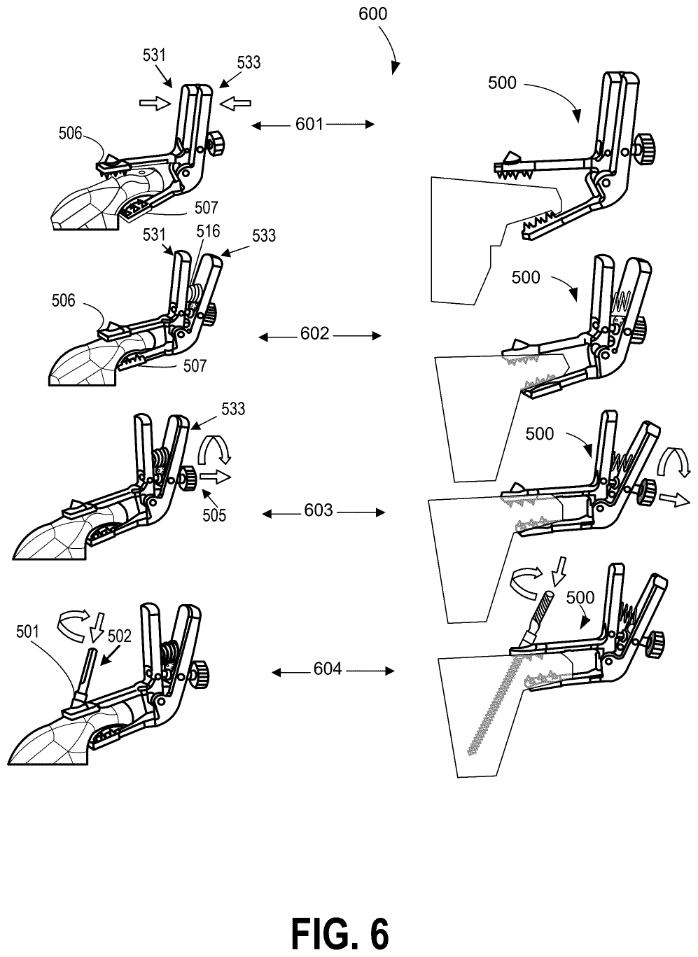

FIG. 6 illustrates an example procedure 600 demonstrating how the preloaded clamp 500 may be applied onto a coracoid process (e.g., the coracoid process 210). The procedure 600 may include various steps. At diagram 601, a practitioner may press the clamp and bring the clamp over the coracoid process. When the superior handle 531 and the inferior handle 533 are pressed together or being squeezed, the preloaded spring (not shown) may be squeezed and compressed, and thus the potential energy of the preloaded spring (not shown) may increase. As the pressure applied on the superior handle 531 and the inferior handle 533 increases, the distance between the superior handle 531 and inferior handle 533 may decrease. The increased pressure between the superior handle 531 and inferior handle 533 may cause the clamping tool to open. In other words, the increased pressure may cause the distance between the superior clamp foot 506a and the inferior clamp foot 507 to increase because the superior handle 531a is connected to the superior clamp foot 506 and the inferior handle 533 is connected to the inferior clamp foot 507. The open preloaded clamp 500 with pressure on the superior handle 531 and the inferior handle 533 may be brought over to a bone, for example the coracoid process.

At diagram 602, the location of the preloaded clamp may be fine tuned. Once a desired location of the coracoid process to secure the preloaded clamp 500 is confirmed, the pressure on the superior handle 531 and the inferior handle 533 may then be released. The compressed preloaded spring 516 may have potential energy. As the pressure is released, the preloaded spring 516 may expand and exert pressure on the superior handle 531 and the inferior handle 533 causing the distance between the superior handle 531 and the inferior handle 533 to increase. The distance between the superior clamp foot 506 and the inferior clamp foot 507 may decrease because the superior handle 531 is connected to the superior clamp foot 506 and the inferior handle 533 is connected to the inferior clamp foot 507. The superior clamp foot 506 and the inferior clamp foot 507 may become in contact with the coracoid process and squeeze the coracoid process because the preloaded spring 516 expands. The superior clamp foot 506 and the inferior clamp foot 507 may clamp onto the coracoid process and the preloaded clamp 500 may be held in place by the preloaded spring 516.

At diagram 603, once adequate resistance is felt on the screw 505, the rotation of the tightening screw 505 may be stopped. The tightening screw 505 may then be pulled outwards away from the inferior handle 533 to cause the tightening screw 505 to lock in place. Once tightening screw 505 is locked in place, the movement of the tightening screw 505 may be restricted and may not be further rotated.

At diagram 604, once the position of the preloaded clamp is confirmed, the bone screw 502 may then be inserted into the coracoid process through the hole 501 to further restrict the movement of the preloaded clamp 500. The bone screw 502 may provide the preloaded clamp 500 additional anchorage onto the coracoid process. This quick and simple way of attachment may allow a quick, temporary fixation of the clamp to the coracoid process. Clamping the clamping tool onto the coracoid process by this preload may enable the practitioner to use both hands for the screw fixation. This convenience may be achieved when the clamp is not held in the other hand during screw attachment.

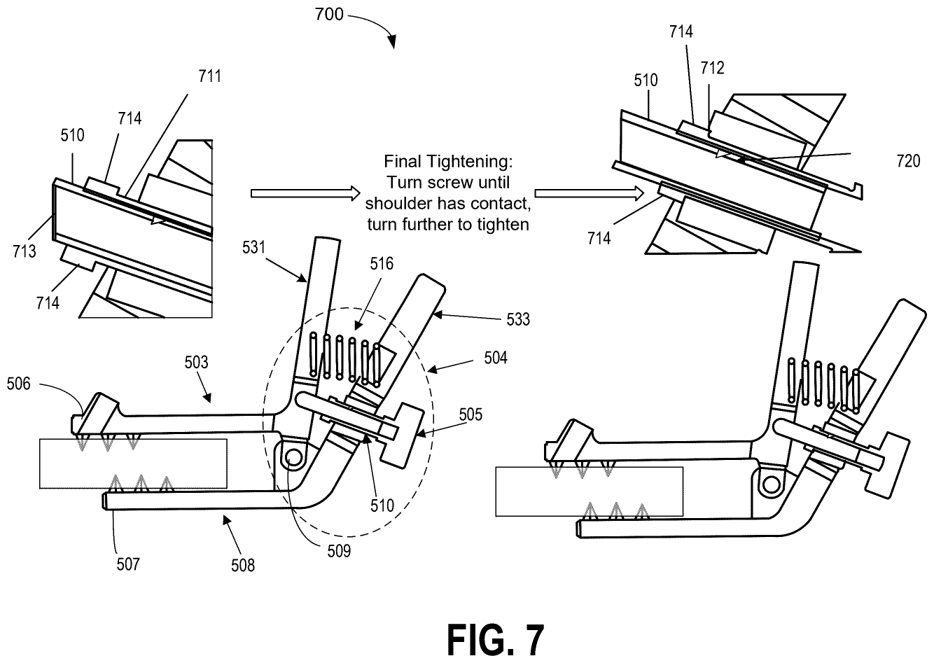

FIG. 7 illustrates a diagram 700 that illustrates an example of how the tightening screw 505 of the clamp closing mechanism 504 is tightened to secure the clamping tool 500 about the coracoid process.

The inferior leg 508 may have a tightening hole 510. The tightening hole 510 may be on the opposite side of the inferior clamp foot about the hinge 509. The closing mechanism may include the tightening screw 505. The hinge 509 may be offset from the tightening screw 505. The tightening hole 510 may be configured to receive the tightening screw 505. The tightening screw 505 may be configured to tighten the superior clamp foot 506 and the inferior clamp foot 507 about the coracoid process 210.

The tightening screw 505 may be placed inside the tightening hole 510. The tightening screw 505 may contain a shaft 713 and a shell 711. The shaft 713 of the tightening screw and the shell 711 of the tightening screw may form a stopper mechanism, such as via an extended shoulder 714 of the shell 711. The head of the tightening screw 505 may be part of the shell 711 and/or the body of the tightening screw may be part of the shaft 713. An end of the shell may be the head of the tightening screw 505 and/or an opposite end of the shell 711 may be a shoulder 714. The head of the tightening screw 505 and the shoulder 714 may be located on the opposite sides of the inferior leg 507 through the tightening hole 510. The shoulder 714 may slide relative to the body of the tightening screw. Before final tightening, there may be a gap at 712 between the inferior leg 507 and the shoulder 714. The final tightening procedure may be performed by turning the tightening screw 505 until the clamp feet and the bone are in contact, and then turning to further tighten the tightening screw 505 until the shoulder 714 is pulled away from the superior leg along the slide movement so that the shoulder 714 is in contact with the inferior leg, or there is no gap between the inferior leg 507 and the shoulder 714. After the final tightening procedure, there is no gap at 712 between the inferior leg and the shoulder 714. Accordingly, after the final procedure, the clamping tool 500 is secured about the coracoid process.

FIG. 8A illustrates an example of a clamping tool 800 that includes a removable superior foot 820. The clamping tool 800 may be similar to the clamping tool 500. However, the clamping tool 800 may include a removable superior clamp foot 820. In some examples, the clamping tool 800 may be smaller than the clamping tool 500 as a whole. After a final fixation, the superior clamp foot 820 may be fixed onto the coracoid process using one or more screws, and a remainder of the clamping tool 800 may be removed. Leaving the superior clamp foot 820 on the coracoid process without the rest of the clamping tool 800 may increase the visual coverage of a surgical field and decrease the interference of clamping tool 800 with surgical instruments.

The clamping tool 800 may include a superior leg 803 and an inferior leg 808. The superior leg 803 and the inferior leg 808 may be fixed via a clamp closing mechanism 804 of the clamping tool 800. The superior leg 803 may include a removable superior foot 820. The superior leg 803 may extend from a proximal end, which is coupled to the clamp closing mechanism 804, to a distal end.

The superior leg 803 may include a superior clamp foot 820 that is located at the distal end of the superior leg 803 with respect to the closing mechanism 804. A superior handle 831 may be connected to the superior leg 803 at the closing mechanism 804. The superior clamp foot 820 may include the removable superior foot 820 and a clamp foot base 806b. The removable superior foot 820 and the clamp foot base 806b may form a mortise and tenon joint like connection. The clamp foot base 806b may be shaped like a mortise 806c and the removable superior foot 820 may be shaped like a tenon 806a. The tenon 806a of the removable superior foot 820 may be fitted into the mortise 806c of the clamp foot base 806b. The tenon like removable superior foot 820 may be slid into the clamp foot base 806b during a fixation process. In the illustrated example, the removable superior foot 820 may be fitted into the clamp foot base 806b through a fork shape with a dovetail interface connection.

An inner surface 826 of the removable superior foot 820 may be configured to contact a superior surface of the coracoid process (e.g., the superior surface 202 of the coracoid process 210) when the clamping tool 800 is being used during a surgical procedure. For example, the removable superior foot 820 may include clamp teeth 814 that extend from the inner surface 826 of the removable superior foot 820. In some examples, the removable superior foot 820 may include a plurality of clamp teeth 814 (e.g., six clamp teeth) that are configured to engage the superior surface of the coracoid process to secure the superior leg 803 to the coracoid process. As described in more detail herein, in other examples, the removable superior foot 820 may take different shapes and/or include a different number of clamp teeth 814.

The inferior leg 808 may extend from a proximal end, which is coupled to the clamp closing mechanism 804, to a distal end. An inferior handle 833 may be connected to the inferior leg 808 at the closing mechanism 804. The inferior leg 808 may include an inferior clamp foot 807 at the distal end of the inferior leg 808 with respect to the closing mechanism 804. An inner surface 827 of the inferior clamping foot 807 may be configured to contact an inferior surface of a coracoid process (e.g., the inferior surface 204 of the coracoid process 210) when the clamping tool 800 is being used during a surgical procedure. For example, the inferior clamp foot 807 may include clamp teeth 815 that extend from the inner surface 827 of the inferior clamping foot 807. For example, the inferior clamp foot 807 may include a plurality of clamp teeth 815 (e.g., four clamp teeth) that are configured to engage the inferior surface of the coracoid process to secure the inferior leg 808 to the coracoid process. In other examples, the inferior clamp foot 807 may take different shapes and/or include a different number of clamp teeth 815.

In some examples, the superior leg 803 and/or the inferior leg 808 may be bent or curved. In other examples, the superior leg 803 and/or the inferior leg 808 may be relatively straight. Further, in some examples, the superior leg 803 may be longer than the inferior leg 808. For example, the distance between an end of the superior clamp foot 806 and the closing mechanism 804 may be greater than an end of the inferior clamp foot 807 and the closing mechanism 804.

The removable superior foot 820 may include one or more holes, such as hole 801a and hole 801b. The holes 801a and 801b may be configured to receive bone screws 802a and 802b. The bone screw 802a and/or 802b may be configured to affix the removable superior foot 820 to the coracoid process. The hole 801a in the removable superior foot 820 may be at an angle with respective to the removable superior foot. The hole 801b in the removable superior foot may be at another angle with respective to the removable superior foot 820. The angle between the removable superior foot 820 and the hole 801a may be different from the angle between the removable superior foot 820 and the hole 801b. For example, the holes 801a and 801b may be pass through holes that penetrate through the removable superior foot 820 at angles to allow the bone screws 802a and 802b to pass from an outer surface 828 of the removable superior foot 820 to the inner surface 826 of the removable superior foot 820 to secure the removable superior foot 820 to the superior surface of the coracoid process. The two holes 801a and 801b with two bone screws 802a and 802b of the removable superior foot 820 may provide strong anchorage to securely fixate the removable superior foot 820 onto the coracoid process, especially when the removable superior foot 820 is fixated onto the coracoid process alone without the remainder of the clamping tool 800. The different angles of the bone screws 802a and 802b may further restrict the movement of the removable superior foot 820.

The holes 801a and 801b may contain corresponding internal threads to facilitate the receipt of the bone screws 802a and 802b. The thread of the hole 801a and the thread of the bone screw 802a may be used to tighten the fixation between the removable superior foot 820 and the bone screw 802a. Similarly, the thread of the hole 801b and the thread of the bone screw 802b may be used to tighten the fixation between the removable superior foot 806 and the bone screw 802b. When the bone screws 802a and 802b are fixated to the removable superior foot 820, the heads 812a and 812b of the bone screw may be exposed at the outer surface 828 of the removable superior foot 820 and the bodies 811a and 811b of the bone screw may be exposed at through the inner surface 826 of the removable superior foot 820. The head 812a and 812b of the bone screws may be textured (e.g., to improve a grip on the bone screw).

As noted above, the clamping tool 800 may include a closing mechanism 804 that is coupled at a proximate end of the superior leg 803 and at a proximate end of the inferior leg 808. The closing mechanism 804 may include a hinge 809, and a tightening screw 805. The hinge 309 may be offset from the tightening screw 305. The hinge 309 may be made of various materials, such as metal, plastic, or steel. The hinge 809 may connect to the proximate ends of the superior leg 803 and the inferior leg 808. As described in more detail herein, the superior leg 803 and the inferior leg 808 may pivot relative to each other about the hinge 809. As such, the hinge 809 may provide a degree of freedom between the movement of the superior leg 803 and inferior leg 808. As described in more detail herein, when the superior leg 803 and the inferior leg 808 move with respect to each other about the hinge 809, the distance between the clamp foot base 806b and inferior clamp foot 807 may become larger or smaller.

The clamping tool 800 may include a preloaded spring 816. One end of the preloaded spring 816 may be connected to the superior handle 831 and an opposite end of the preloaded spring 816 may be connected to the inferior handle 833. The superior handle 831 may be located on an opposite side of the hinge 809 with respect to the clamp foot base 806b. The inferior handle 833 may be located on an opposite side of the hinge 809 with respect to the inferior clamp foot 807.

The clamping tool 800 may allow a quick and simple way of attachment. At a preload state, the preloaded spring 816 may be preloaded with potential energy. The preloaded spring 816 may push the superior handle 831 and the inferior handle 833 apart to cause the clamp foot base 806b and the inferior clamp foot 807 to be closer to each other. When the superior handle 831 and the inferior handle 833 are pressed together or being squeezed, the preloaded spring 816 may be squeezed and compressed, and thus the potential energy of the preloaded spring 816 may increase. As the pressure applied on the superior handle 831 and the inferior handle 833 increases, the distance between the superior handle 831 and inferior handle 833 may decrease. The increased pressure between the superior handle 831 and inferior handle 833 may cause the clamping tool to open. In other words, the increased pressure may cause the distance between the clamp foot base 806b and the inferior clamp foot 807 to increase because the superior handle 831 is connected to the clamp foot base 806b and the inferior handle 833 is connected to the inferior clamp foot 807. The clamping tool 800 with pressure on the superior handle 831 and the inferior handle 833 may be brought over to a bone, for example the coracoid process. Once a desired location to secure the preloaded clamp 800 is confirmed, the pressure on the superior handle 831 and the inferior handle 833 may then be released. The compressed preloaded spring 816 may have potential energy. As the pressure is released, the preloaded spring 816 may expand and exert pressure on the superior handle 831 and the inferior handle 833 causing the distance between the superior handle 831 and the inferior handle 833 to increase. The distance between the clamp foot base 806b and the inferior clamp foot 807 may decrease because the superior handle 831 is connected to the clamp foot base 806b and the inferior handle 833 is connected to the inferior clamp foot 807. The clamp foot base 806b and the inferior clamp foot 807 may become in contact with the coracoid process and squeeze the coracoid process because the preloaded spring 816 expands. The clamp foot base 806b and the inferior clamp foot 807 may clamp onto the coracoid process and the clamping tool 800 may be held in place by the preloaded spring 816.

The tightening screw 805 may include a head and a body portion. A proximate end of the body portion may terminate at the head of the tightening screw, while a distal end (e.g., point) of the body portion may be configured to contact the superior leg 803. For example, the distal end, or point, of the body portion of the tightening screw 805 may be secured to the proximate end of the superior leg 803, such that the head of the tightening screw moves toward the hinge 809 when rotated clockwise. As described herein, as the head of the tightening screw 805 is rotated clockwise, the point of the body portion may cause the superior leg 803 to pivot about the hinge 809 to cause the clamp foot base 806b to move closer to the inferior clamp foot 807 (e.g., cause the superior and inferior clamp feet to close). As the head of the tightening screw 805 is rotated counterclockwise, the point of the body portion may cause the superior leg 803 to pivot about the hinge 809 to cause the clamp foot base 806b to move away from to the inferior clamp foot 807 (e.g., cause the superior and inferior clamp feet to open).

When the tightening screw 805 is positioned through the inferior leg 808, the body portion of the tightening screw 805 may be in contact with the superior leg 803, and the head of the tightening screw 805 may be exposed at the opposite side of the inferior leg 808. In some examples, the tightening hole 810 may contain internal threads to facilitate the receipt and movement of the threaded body portion of the tightening screw 805. The head of the tightening screw 805 may be textured (e.g., to improve a grip on the tightening screw).

As noted above, as the head of the tightening screw 805 is rotated clockwise, the point of the tightening screw 805 may cause the superior leg 803 to pivot about the hinge 809 to cause the superior clamp foot 806b to move closer to the inferior clamp foot 807. For instance, as the head of the tightening screw 805 is rotated clockwise, the tightening screw 805 may push the superior leg 803 forwards to cause the superior leg 803 to pivot about the hinge 809 to cause the clamp foot base 806b to move towards the inferior clamping foot 807. This clockwise rotation of the head may be performed to tighten the superior and inferior clamping feet about the coracoid process. The tightening screw 805 may restrain movement of superior leg 803 and inferior leg 808 in order to maintain pressure of surfaces removable superior foot 820 and inferior clamp foot 807 upon the coracoid process.

As the head of the tightening screw 805 is rotated counterclockwise, the point of the body portion of the tightening screw 805 may cause the superior leg 803 to pivot about the hinge 809 to cause the clamp foot base 806b to move away from the inferior clamping foot 807. For instance, as the head of the tightening screw 805 is rotated counterclockwise, the tightening screw 805 may pull the superior leg 803 backwards to cause the superior leg 803 to pivot about the hinge 809 to cause the clamp foot base 806b to move away from the inferior clamping foot 807. This counterclockwise rotation of the head may be performed to loosen the superior and inferior clamping fect and allow the clamping tool 800 to be removed from the coracoid process.

The body 811a and/or 811b of the bone screws 802a and 802b may be embedded in a bone (e.g., either in hole(s) separately drilled or may be impacted into an unprepared bone) through the removable superior foot 820 of the clamping tool 800. The bone screws 802a and 802b may be positioned through the removable superior foot 820.

In an example, a tracking system may be used to track the relative position and orientation of surgical instrument(s) and patient anatomical structure(s) for surgical navigation. The tracking system may include a navigation sensor (not shown) (e.g., optical sensor/camera, electromagnetic sensor, etc.) for determining the position and orientation of one or more navigation arrays (not shown) that include one or more markers, for example, optical markers, electromagnetic markers, or other types of markers. An array coupling interface/component 830 may be configured to mate with a corresponding mating interface on a navigation array (not shown) to fix the navigation array to the clamping tool 800 to provide an anatomical reference point for a tracking system providing surgical navigation. Examples of the array coupling interface/component 830 are described in greater detail in U.S. Pat. No. 11,644,053, published May 27, 2021, entitled INSTRUMENT COUPLING INTERFACES AND RELATED METHODS, the entire disclosure of which is hereby incorporated by reference. For example, in U.S. Pat. No. 11,644,053, the array coupling interface may include a coupling that can mate a first object (e.g., an instrument adapter, such as the removable superior foot 820) to a second object (e.g., a navigation array 840). Referring to U.S. Pat. No. 11,644,053, the navigation array 304 may be an example of a navigation array 840 that can be coupled to the array coupling interface 830 of the clamping tool 800, and the coupling 308 may be an example of the array coupling interface 830 of the clamping tool 800. For instance, the clamping tool 800 may include a first coupling component, such as the array coupling interface/component 830 (e.g., the first coupling component 310 of U.S. Pat. No. 11,644,053) that is associated with the first object (e.g., the removable superior foot 820), and a second coupling component, such as the coupling interface of the navigational array 842 (e.g., the second coupling component 312 of U.S. Pat. No. 11,644,053) that associated with the second object (e.g., the navigation array 840). Further, and for example, the array coupling interface 830 may include a V-shaped protrusion (e.g., that is part of the array coupling interface/component 830) and a corresponding V-shaped recess (e.g., that is part of the navigation array), for instance, as described by the respective first and second components 102A, 102B in U.S. Pat. No. 11,644,053.

Some other examples of the array coupling interface/component 830 are described in greater detail in U.S. Pat. No. 10,731,687, published May 23, 2019, entitled INSTRUMENT COUPLING INTERFACES AND RELATED METHODS, the entire disclosure of which is hereby incorporated by reference. For example, in U.S. Pat. No. 10,731,687, an array coupling interface may include a coupling that can mate a first object (e.g., an instrument adapter, such as the removable superior foot 820) to a second object (e.g., a navigation array 840). Referring to U.S. Pat. No. 10,731,687, the navigation array 300 may be an example of a navigation array 840 that can be coupled to the array coupling interface 830 of the clamping tool 800, and the coupling 200 may be an example of the array coupling interface 830 of the clamping tool 800. For instance, the clamping tool 800 may include a first coupling component, such as the array coupling interface/component 830 (e.g., the first coupling interface 202A, 202A′, or 202A″ of U.S. Pat. No. 10,731,687) that is associated with the first object (e.g., the removable superior foot 820), and a second coupling component, such as the coupling interface of the navigational array 842 (e.g., the second coupling interface 202B of U.S. Pat. No. 10,731,687) that associated with the second object (e.g., the navigation array 840). Further, and for example, the array coupling interface 830 may include a rotation stop protrusion (e.g., that is part of the array coupling interface/component 830) and a corresponding rotation stop recess (e.g., that is part of the navigation array), for instance, as described by the respective first and second coupling interfaces 202A, 202B in U.S. Pat. No. 10,731,687. In another example, the array coupling interface 830 may include a V-shaped protrusion (e.g., that is part of the array coupling interface/component 830) and a corresponding V-shaped recess (e.g., that is part of the navigation array), for instance, as described by the respective first and second components 102A, 102B in U.S. Pat. No. 10,731,687.

Some additional examples of the array coupling interface/component 830 are described in greater detail in U.S. Pat. No. 10,722,223, published Dec. 6, 2018, entitled COUPLING DEVICES FOR SURGICAL INSTRUMENTS AND RELATED METHODS, the entire disclosure of which is hereby incorporated by reference. For example, in U.S. Pat. No. 10,722,223, an array coupling interface may include a coupling that can mate a first object (e.g., an instrument adapter, such as the removable superior foot 820) to a second object (e.g., a navigation array 840). Referring to U.S. Pat. No. 10,722,223, the navigation array 12 may be an example of a navigation array 840 that can be coupled to the array coupling interface 830 of the clamping tool 800, and the coupling device 100 may be an example of the array coupling interface 830 of the clamping tool 800. For instance, the clamping tool 800 may include a first coupling component, such as the array coupling interface/component 830 (e.g., the instrument as shown in FIG. 5A of U.S. Pat. No. 10,722,223) that is associated with the first object (e.g., the removable superior foot 820), and a second coupling component, such as the coupling interface of the navigational array 842 (e.g., the instrument with a recess mating geometry as shown in FIG. 5B of U.S. Pat. No. 10,722,223) that associated with the second object (e.g., the navigation array 840). Further, and for example, the array coupling interface 830 may include a protrusion (e.g., that is part of the array coupling interface/component 830) and a corresponding recess (e.g., that is part of the navigation array), for instance, as described by the instrument mating geometry as shown in FIGS. 5A and 5B of U.S. Pat. No. 10,722,223.

FIG. 8B illustrates a diagram that illustrates an example procedure 850 of using a clamping tool with a removable superior foot in a surgical procedure for navigation assistance. The procedure 850 may be performed by a practitioner and a navigation system to perform a surgical procedure using the clamping tool 800 of FIG. 8A. Although described with reference to the clamping tool 800, the procedure 850 may be performed using any of the clamping tools that are described herein and that are configured to be coupled to a navigation array.

At 851, the practitioner may attach the array coupling interface/component 830 to the removable superior foot 820. At 852, the practitioner may mate the array coupling interface/component 830 with a corresponding mating interface on a navigation array to fix the navigation array to the clamping tool 800, for example, to provide an anatomical reference point for a tracking system providing surgical navigation.

At 853, the navigation array may be tracked by a navigation sensor. The clamping tool 800 (e.g., the array coupling interface/component 830 with the navigation array attached) and the navigation sensor may be used throughout a surgical procedure. The navigation sensor may track the navigation array on the clamping tool 800 to intra-operatively establish the position and orientation of the patient anatomy and guide surgical steps in the desired position, orientation and/or trajectory according to a surgical plan.

Data may be collected using the navigation array and navigation sensor and processed using a processing device with a memory or a storage device to register the patient anatomy to corresponding points in pre-operative imaging or models associated with a surgical plan. Further, intraoperative data may be collected using the navigation array and the navigation sensor to track the relative position and orientation of surgical instruments and patient anatomical structure(s) to provide surgical navigation in accordance with a surgical plan.

At 854, during a surgical procedure, a computer may track a position of the navigation array on the clamping tool 800 fixed to the patient anatomy during a surgical procedure. The computer may initiate the tracking of the navigation array continuously and automatically. During a surgical procedure, many events may occur (e.g., patient movement, instrument movement, loss of tracking, etc.) that may disturb the tracking process. The computer may be implemented to verify and adjust tracking parameters continuously or periodically. For example, the computer may continuously track a position of the navigation array that is attached to the clamping tool 800 and utilize the tracked position to guide surgical steps. For instance, the computer may track the position of the navigation array to assist in the navigation of surgical instruments (e.g., the clamping tool 800) and/or implants to ensure the surgical instruments are in the desired position, orientation and/or trajectory relative to the patient. The computer may use surgical assistance systems such as surgical robotic systems, surgical augmented and/or virtual reality systems, or the like to ensure that the surgical instruments are in the desired position, orientation and/or trajectory relative to the patient.

FIG. 8C illustrates an example surgical procedure 860 with navigational assistance. At 861 a user may attach a clamping tool to a coracoid process. At 862 the user may attach a navigational sensor (e.g., navigational tracker) to the clamping tool, for example after the clamping tool is attached to the coracoid process. At 863 the user may drill and/or ream a hole (e.g., cavity) in a glenoid. At 864, the user may track a position and/or movement of the clamping tool and/or the navigational sensor (e.g., navigational tracker), for example during the drilling and/or reaming of the hole (e.g., cavity) in the humeral bone.

FIG. 8D illustrates another example surgical procedure 870 with navigational assistance. At 871 a user may attach a navigational sensor (e.g., navigational tracker) to a surface of a glenoid. At 872 the user may drill and/or ream a hole (e.g., cavity) in the glenoid. At 874 the user may track a position and/or movement of the clamping tool and/or the navigational sensor (e.g., navigational tracker), for example during the drilling and/or reaming of the hole (e.g., cavity) in the glenoid. At 875 the user may place an implant in the hole (e.g., cavity) in the glenoid, for example at least partially.

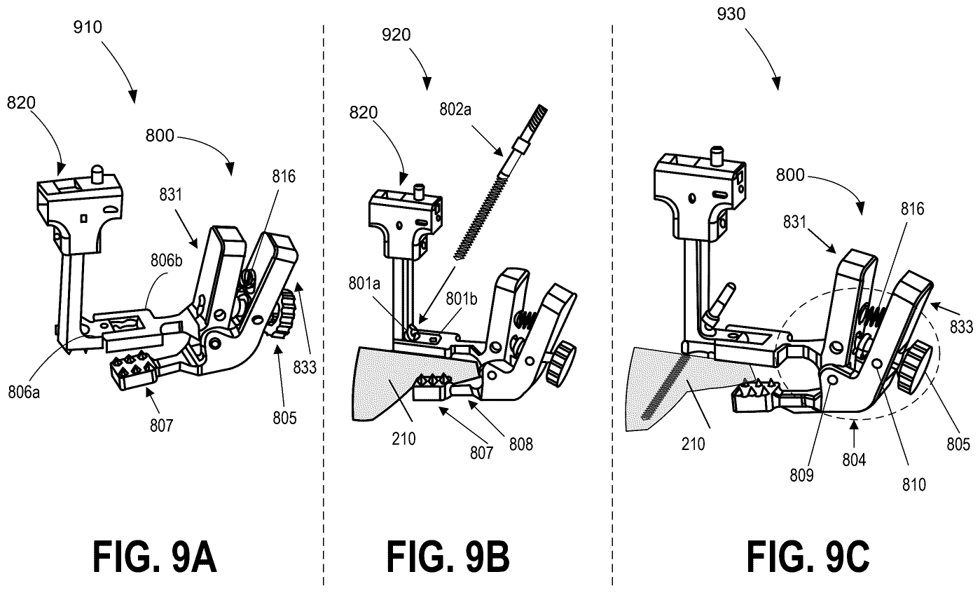

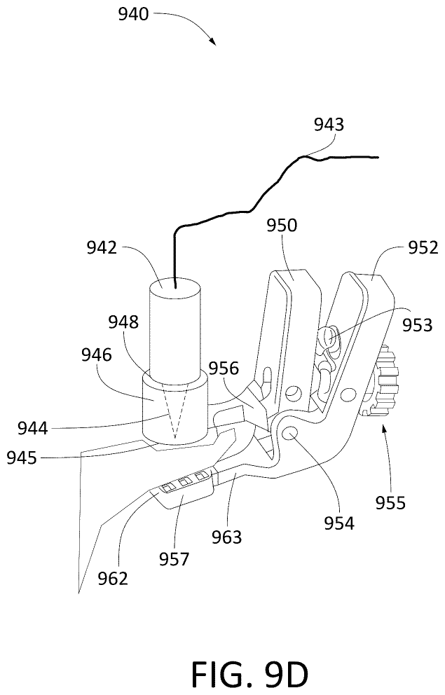

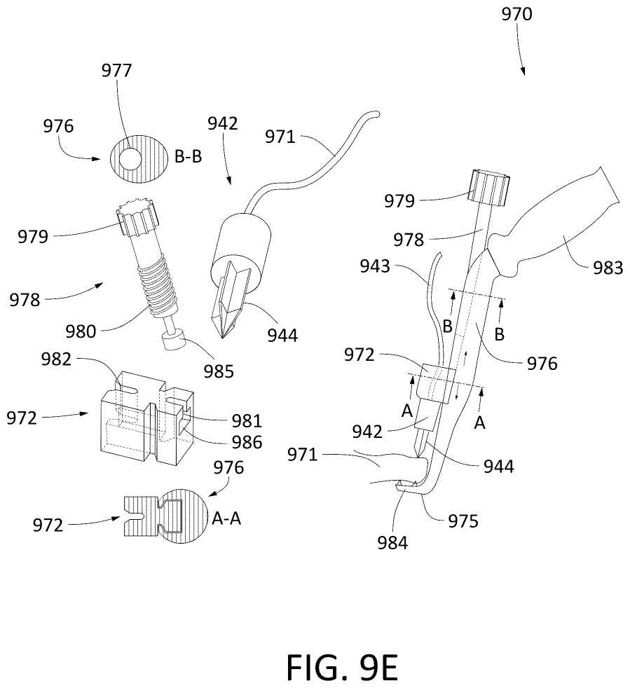

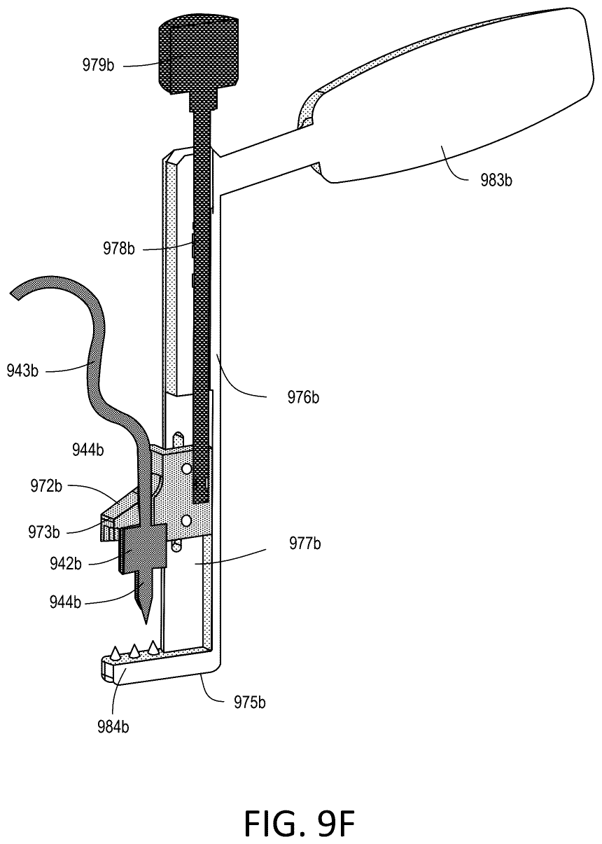

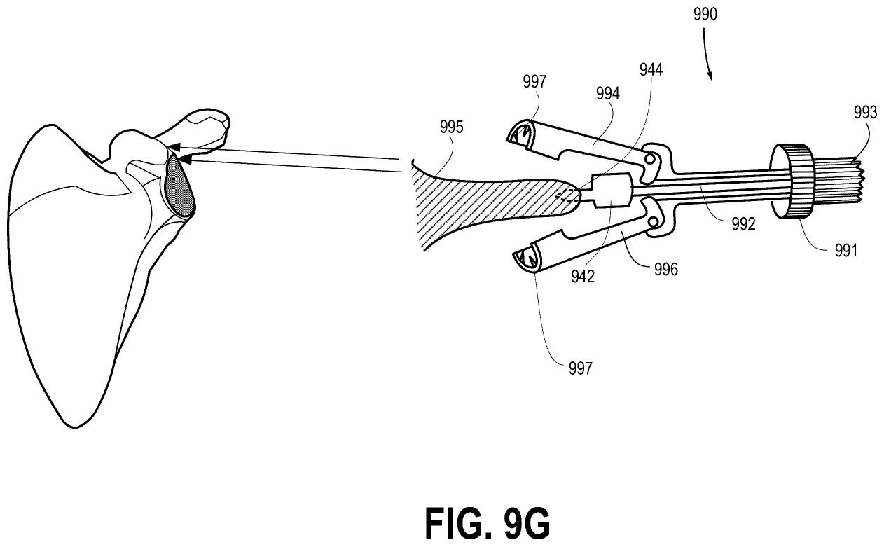

FIGS. 9A-E illustrates an example procedure 900 demonstrating how the clamping tool 800 with the removable superior foot clamp 820 may be applied onto a coracoid process. At 910, the removable superior foot 820 may be slid into the clamp foot base 806b, and the removeable superior foot 820 may be attached to the superior leg 803. The practitioner may press clamp and bring the removable superior foot clamp to the coracoid process. As noted above, when the superior handle 831 and the inferior handle 833 are pressed together or being squeezed, the preloaded spring 816 may be squeezed and compressed, and thus the potential energy of the preloaded spring 816 may increase. As the pressure applied on the superior handle 831 and the inferior handle 833 increases, the distance between the superior handle 831 and inferior handle 833 may decrease. The increased pressure between the superior handle 831 and inferior handle 833 may cause the clamping tool 800 to open. In other words, the increased pressure may cause the distance between the clamp foot base 806b and the inferior clamp foot 807 to increase because the superior handle 831 is connected to the clamp foot base 806b and the inferior handle 833 is connected to the inferior clamp foot 807. The clamping tool 800 with pressure on the superior handle 831 and the inferior handle 833 may be brought over to a bone, for example the coracoid process.

Once a desired location to secure the clamping tool 800 is confirmed, the pressure on the superior handle 831 and the inferior handle 833 may then be released. The desired location of the clamping tool 800 may be fine tuned by slightly move the released clamping tool 800. The compressed preloaded spring 816 may have potential energy. As the pressure is released, the preloaded spring 816 may expand and exert pressure on the superior handle 831 and the inferior handle 833 causing the distance between the superior handle 831 and the inferior handle 833 to increase. The distance between the clamp foot base 806b and the inferior clamp foot 807 may decrease because the superior handle 831 is connected to the clamp foot base 806b and the inferior handle 833 is connected to the inferior clamp foot 807. The clamp foot base 806b and the inferior clamp foot 807 may become in contact with the coracoid process and squeeze the coracoid process because the preloaded spring 816 expands. The clamp foot base 806b and the inferior clamp foot 807 may clamp onto the coracoid process and the clamping tool 800 may be held in place by the preloaded spring 816. Once adequate resistance is felt on the screw 805, the rotation of the tightening screw 805 may be stopped. The tightening screw 805 may then be pulled outwards away from the inferior handle 833 to cause the tightening screw 805 to lock in place. Once tightening screw 805 is locked in place, the movement of the tightening screw 805 may be restricted and may not be further rotated.

At 920, the practitioner may tighten the clamp by turning the tightening screw 805 until adequate resistance is felt. Once the position is confirmed, the practitioner may insert the bone screws for additional anchorage. The bone screws of the removable superior foot 820 may provide strong anchorage to securely fixate the removable superior foot 820 onto the coracoid process, especially when the removable superior foot 820 is fixated onto the coracoid process alone without the body of the clamping tool 800. The different angles of the bone screws may further restrict the movement of the removable superior foot 820. Clamping the clamping tool 800 onto the coracoid process may enable the practitioner to use both hands for the screw fixation. The practitioner may not need to hold the clamping tool 800 in one hand and fix the bone screw 802a using another hand.

After a final fixation, at 930, the practitioner may separate the superior clamp foot 820 from the clamping tool 800. As such, the superior clamp foot 820 may be fixed onto the coracoid process without the body of the clamping tool 800. Leaving the superior clamp foot 820 onto the coracoid process alone without the body of the clamping tool 800 may increase the visual coverage of a surgical field and decrease the interference of clamping tool with surgical instruments.

A navigational tracker (e.g., as herein) may be placed, for example to enable tracking of a clamping tool and/or a surgical instrument. For example, the navigational tracker may be used for tracking of a clamping tool relative the patient (e.g., bone). The clamping tool may be attached to the navigational tracker and/or may be used to place the navigational tracker near a bone or to attach the navigational tracker to a bone. The navigational tracker (e.g., navigational tracker) 1620 may include an optical sensor/camera, electromagnetic sensor, a navigation array (e.g., navigation array 840, 842), a mini tracker sensor, and/or an optical marker. For example, an optical marker may include an array of optical markers.

The navigational tracker may be placed at a coracoid process (e.g., 1614) and/or at a glenoid cavity (e.g., 1610) for example. The navigational tracker may be placed at an upper portion (e.g. near a supraglenoid tubercle) of a glenoid cavity, such that the navigational tracker does not interfere with a surgical procedure. The navigational tracker may be placed (e.g., at the coracoid process and/or at the glenoid) using bone screw(s). Additionally, or alternatively, the navigational tracker may be placed (e.g., at the coracoid process and/or at the glenoid) less traumatically and/or less invasively than by securing with a screw. For example, the navigational tracker may be placed at a bone without impaction of the bone. The navigational tracker may be placed using a clamping tool (e.g., as herein) and/or may be pushed into a bone (e.g., using a pin).

Surgical navigation, for example using a tracking system (e.g., as herein) may assist with drilling, reaming, placement of a clamping tool, and/or placement of an implant. Surgical navigation may be performed such that there is not interference of a navigational tracker with the surgical procedure. For example, a location of a navigational tracker may be planned (e.g., pre-operatively) such that sufficient space is available for a surgeon to perform the surgical procedure. Additionally, or alternatively, a navigational tracker may be placed less invasively. A surgeon may (e.g., therefore) move the location of the navigational tracker, for example to obtain more space and/or a better of a surgical site. For example, the surgeon may decide to move a clamping tool from a glenoid cavity to a coracoid process (e.g., before and/or during a surgical procedure).

Example clamping tools may be configured to place (e.g., attach) a larger navigational tracker to a bone. For example, a larger navigational tracker may include an infrared (IR) stereovision navigation array. Navigational trackers (e.g., larger navigational trackers) may include a significant mass, which for example may induce significant forces due to gravity and/vibration. A stronger placement may (e.g., therefore be utilized) for a larger navigational tracker, for example supported by a screw (e.g., bone screw). However, for smaller navigation sensors alternative placement mechanisms may be utilized. For example, a clamping tool configured to place the navigational tracker with less bone trauma (e.g., without drilling and/or screwing) may be utilized. The placement mechanism may be configured to place the navigational tracker in a rigid and/or non-toggling connection. The navigational tracker may remain stable throughout a surgical procedure. For example, a clamping tool may place a navigational tracker with less bone trauma (e.g., without drilling and/or screwing) at the coracoid process.

Clamping tools are disclosed, for example where a foot may connected to a bone. A clamping tool may not utilize a bonescrew (e.g., 502) in some examples. For example, a clamping tool may include a mechanism (e.g., on a superior clamp) configured to receive a navigational tracker.

FIG. 9D illustrates an example clamping tool 940. The clamping tool 940 may include a superior leg 956 and an inferior leg 963. The superior leg 956 and the inferior leg 963 may be fixed via a clamp closing mechanism 953 of the clamping tool 940 (e.g., as herein). The inferior leg 963 may include an inferior foot 957. The superior leg 956 may extend from a proximal end, which for example may be coupled to a clamp closing mechanism 953, to a distal end.

The superior leg 956 may include an attachment mechanism 946, for example connected to the superior leg 956. The attachment mechanism 946 may include a cylindrical sleeve configured to receive a navigational tracker 942. The navigational tracker 942 may include a cable 943, for example attached at an end of the navigational tracker 942. The cable 943 may be connected to a device, for example such that power may be received at the navigational tracker 942. Additionally, or alternatively, the cable 943 may be configured to provide data to the navigational tracker 942 and/or receive data from the navigational tracker 942. For example, the data may be tracking data. Additionally, or alternatively, the navigational tracker 942 may include an internal power source, for example a battery and/or a capacitor. The navigational tracker 942 may include a wireless communication device, for example a transceiver. The wireless communication device may be configured to provide data from the navigational tracker 942 (e.g., to the device) and/or receive data at the navigational tracker 942 (e.g., from the device).

The clamping tool 940 may include a superior handle 950, for example connected to the superior leg 956 (e.g., at the closing mechanism 953). The inferior foot 957 and the attachment mechanism 946 may form a mortise and tenon joint like connection.

A surface of the attachment mechanism 946 may be configured to contact a superior surface of the coracoid process (e.g., the superior surface 202 of the coracoid process 210) when the clamping tool 940 is being used during a surgical procedure. For example, the attachment mechanism 946 may include clamp teeth or ridges, for example that may be disposed on a surface of the attachment mechanism 946. Additionally, or alternatively, a surface of the inferior foot 957 may include clamp teeth 962. The inferior foot 957 and/or the clamp teeth 962 may be configured to contact an inferior surface of the coracoid process (e.g., the inferior surface 204 of the coracoid process 210) when the clamping tool 940 is being used during a surgical procedure.

The clamping tool 940 may include a tightening screw knob 955. The tightening screw knob 955 may be configured such that the closing mechanism 953 (e.g., as herein) closes a space between the inferior foot 957 and the attachment mechanism 946, for example at a coracoid process. The closing mechanism 953 may include an elastic element. For example the elastic element may include an elastic element (e.g., preloaded spring) and/or a hinge. The closing mechanism may be configured to close the clamping tool 940 such that the superior leg 956 and the inferior foot 957 move closer together about a hinge 954. The navigational tracker 942 may be placed with less bone trauma (e.g., without drilling and/or screwing), for example at the coracoid process. The navigational tracker 942 may include a pin 944. The pin may be configured to be inserted into a bone, for example the coracoid process. The clamping tool 940 may be configured to insert the pin into the bone, for example when the tightening screw knob 955 is tightened such that the space between the inferior foot 957 and the attachment mechanism 946 is closed (e.g., decreased).

The pin 944 of the navigational tracker 942 may be disposed in the attachment mechanism 946. For example, the pin 944 may extend into an opening 948 of the attachment mechanism 946. A portion of the pin (e.g., a tip) may extend through a second opening 945 of the navigational tracker 942. The second opening 945 of the attachment mechanism 946 may be proximate the bone (e.g., coracoid process), for example when the clamping tool 940 is in use. The second opening 945 may be proximate the inferior foot 957 and/or the opening 948 may be distal the inferior foot 957.

The navigational tracker may be placed using a clamping tool (e.g., as herein) and/or may be pushed into a bone (e.g., using a pin). FIG. 9E illustrates another example clamping tool 970. The clamping tool 970 may be configured to hold a navigational tracker 942, for example for placement at a bone (e.g., coracoid process 971). The navigational tracker 942 may be configured to be placed at the coracoid process 971 (e.g., the superior surface 202 of the coracoid process 210). The navigational tracker 942 may be connected to a cable 943 (e.g., as herein).

An inferior leg 975 may include an inferior foot 984. The inferior foot may include clamp teeth (e.g., 962). The inferior foot may be configured to contact an inferior surface of the coracoid process (e.g., the inferior surface 204 of the coracoid process 210), for example when the clamping tool 970 is being used during a surgical procedure.

The clamping tool 970 may include an attachment mechanism 972. The attachment mechanism 972 may be disposed on a shaft 976 of the clamping tool 970. For example, the attachment mechanism 972 may be configured to slide along the shaft 976 such that the attachment mechanism 972 may move closer to or further from the inferior foot 984.

FIG. 9E also illustrates a cross-sectional view of the attachment mechanism 972 and the shaft 976 taken along the line A-A. The attachment mechanism 972 is shown connected to the shaft 976. An first aperture 981 of the attachment 972 may be disposed in an interior of the shaft 976. A second aperture 982 of the attachment mechanism 972 may be exterior to the shaft 976. The second aperture 982 of the attachment mechanism 972 may be configured to receive the cable 943, for example attached to the navigational tracker 942.

The first aperture 981 of the attachment mechanism 972 may be configured to receive a plunger 978. The plunger 978 may be disposed, at least partially, in the shaft 976 and/or may be configured to move within the interior of the shaft. For example, the plunger 978 may move closer to or further from the inferior foot 984. The plunger 978 may include a knob 979. When the knob 979 is turned, for example clock-wise, the plunger 978 may rotate such that the plunger 978 moves closer to the inferior foot 984. When the knob 979 is turned, for example counter-clockwise, the plunger 978 may rotate such that the plunger 978 moves further from the inferior foot 984.

FIG. 9E also illustrates a cross-sectional view of the shaft 976 taken along the line B-B. The plunger 978 may include threads 980, for example configured to engage with threads 977 in the interior of the shaft 976. The plunger 978 may be shaped such that the plunger 978 may be received in the first aperture 981 of the attachment mechanism 972. For example, a head 985 of the plunger 978 may be configured to be received in a lower portion 986 of the first aperture 981. The head 985 of the plunger 985 and the lower portion 986 of the first aperture 981 may be configured such that the head 985 of the plunger 978 may not be removed form the lower portion 986 of the first aperture 981 while the plunger 978 is, at least partially, disposed in the shaft 976.

The clamping tool 970 may be configured to place the navigational tracker 942 at a bone (e.g., coracoid process 971), for example when the knob 979 is rotated (e.g., clock-wisc). For example, a user may hold a handle 983 of the clamping tool 970 and/or rotate the knob 979 of the plunger 978. The navigational tracker 942 may be inserted into the bone, for example by rotating the knob 979 of the plunger 978. For example, the pin 944 of the navigational tracker 942 may be pushed into the bone when the knob 979 is rotated (e.g., clock-wise) and the inferior foot 984 abuts the bone.

FIG. 9F illustrates another example clamping tool 970b. The clamping tool 970b may be configured to hold a navigational tracker 942b, for example for placement at a bone (e.g., coracoid process 971). The navigational tracker 942b may be configured to be placed at the coracoid process (e.g., the superior surface 202 of the coracoid process 210). The navigational tracker 942b may be connected to a cable 943b (e.g., as herein).

A leg 975b may include a foot 984b. The foot 984b may include clamp teeth (e.g., 962). The foot 984b may be configured to contact an inferior surface of the coracoid process (e.g., the inferior surface 204 of the coracoid process 210), for example when the clamping tool 970b is being used during a surgical procedure.

The clamping tool 970b may include an attachment mechanism 972b. The attachment mechanism 972b may be disposed on a shaft 976b of the clamping tool 97b. For example, the attachment mechanism 972b may be configured to slide along the shaft 976b such that the attachment mechanism 972b may move closer to or further from the foot 984b. The attachment mechanism 972b may include a contact portion 973b. The contact portion 973b may be configured to hold the navigational tracker 942b. Additionally, or alternatively, the contact portion 973b may be configured to abut the coracoid process (e.g., the superior surface 202 of the coracoid process 210).

The shaft 976b may receive a plunger 978b. The plunger 978b may be disposed, at least partially, in the shaft 976b and/or may be configured to move within the interior of the shaft. For example, the plunger 978b may move closer to or further from the foot 984b. The plunger 978b may include a knob 979b. When the knob 979b is pushed, for example downward, the plunger 978b may move closer to the foot 984b. When the knob is pulled, for example upward, the plunger 978b may move further from the foot 984b. Additionally, or alternatively, the user may turn the knob 979b to actuate movement of the attachment mechanism 972b (e.g., as in FIG. 9E).

An alcove 977b may be disposed in the shaft 976b, for example such that the attachment mechanism 972b may slide within the alcove 977b (e.g., only). Additionally, or alternatively, pins may be disposed in the shaft 977b, for example at an upper portion and at a lower portion of the shaft 977b. Pins may block movement of the attachment mechanism 972b, for example such that the attachment mechanism 977b may move in the alcove 977b (e.g., only).

The clamping tool 970b may be configured to place the navigational tracker 942b at a bone (e.g., coracoid process 971), for example when the knob 979b is pushed (e.g., downward). For example, a user may hold a handle 983b of the clamping tool 970b and/or push the knob 979b of the plunger 978b. The navigational tracker 942b may be inserted into the bone, for example by pushing the knob 979b of the plunger 978b. For example, the pin 944b of the navigational tracker 942b may be pushed into the bone when the knob 979b is pushed (e.g., downward) and the foot 984b and/or the contact portion 973b may abuts the bone. The navigational tracker 942b may snap into the attachment mechanism 972b. For example, there may be two contact portions 973b and the attachment mechanism 972b may snap in between the two contact portions 973b.

The navigational tracker may be placed (e.g., at the coracoid process and/or at the glenoid) laterally. FIG. 9G illustrates an example clamping tool 990. The clamping tool 990 may be configured to attach to a navigational tracker 942. The navigational tracker 942 may be attached to a rod 992 of the clamping tool 990. The rod 992 and/or the navigational tracker 942 may be configured to extend from an actuator 991. For example, the rod 992 and/or the navigational tracker 942 may be configured to extend from the actuator 991 and toward a bone (e.g., coracoid process 995). The rod 992 and/or the navigational tracker 942 may be configured to extend from the actuator 991 when the actuator is turned clock-wise and/or extend toward the actuator 991 when the actuator is turned counter-clockwise. A pin 944 of the navigational tracker 942 may be configured to push into a bone. For example, the pin 944 of the navigational tracker 942 may push into a coracoid process 995 laterally.

The clamping tool 990 may include a turn knob 993. The turn knob 993 may be connected to an arm 994. For example, the turn knob 993 may be connected to two arms 994, 996. The arms 994, 996 may be configured to move closer together or further apart, for example when the turn knob 993 is actuated. The arms 994, 996 may be configured to move closer together (e.g., close) when the turn knob 993 is turned clock-wise and/or move further apart (e.g., open) when the turn knob 993 is turned counter-clockwise. The arms 994, 996 may be configured to contact a bone, for example the coracoid process 995. For example arm 994 may contact the coracoid process 995 (e.g., the superior surface 202 of the coracoid process 210) and/or arm 996 may contact the coracoid process 995 (e.g., the inferior surface 204 of the coracoid process 210) when the clamping tool 990 is being used. Arm 994 and/or arm 996 may include clamp teeth 997, for example at an end of arm 994 and/or arm 996 distal the rod 992. The clamp teeth 997 may be configured to contact the coracoid process 995 when the clamping tool 990 is being used during a surgical procedure. The clamping tool 990 may disconnect from the navigational tracker 942 such that the clamping tool 990 may be removed during a surgical procedure.

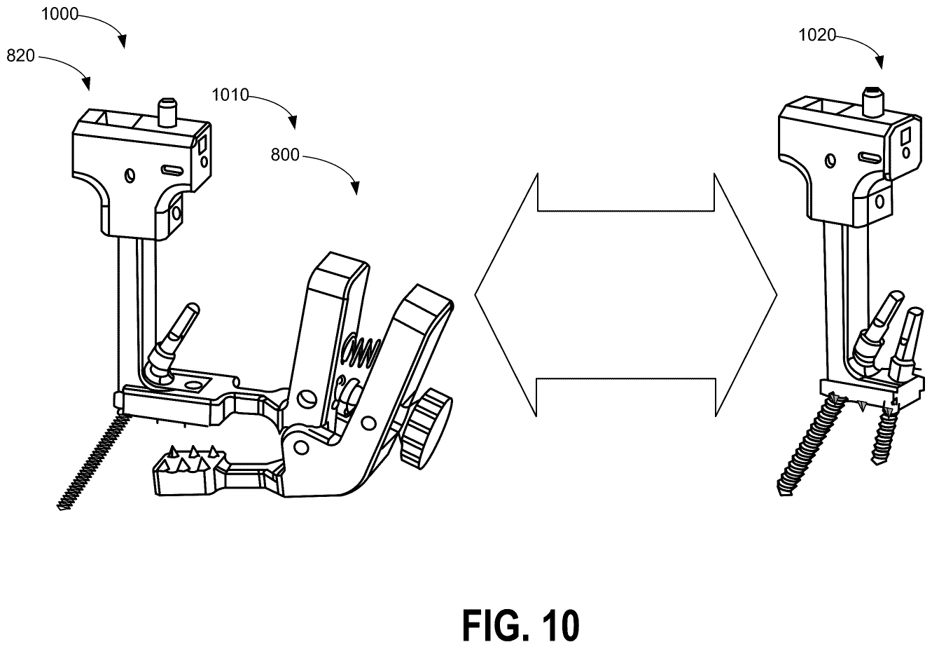

FIG. 10 illustrates two different perspectives of the clamping tool 800. Prior to separation of the superior clamp foot 820, as shown in 1010, both the removable superior foot 820 and the body of the clamping tool 800 may be secured onto the coracoid process, and a bone screw (e.g., a single bone screw) may be inserted through the superior clamp foot 820 and into the coracoid process. In 1020, the superior clamp foot 820 may be separated from the clamp foot base, and the body of the clamping tool may be removed from the superior clamp foot 820. After removal, the practitioner may insert an additional bone screw to more securely fix the superior clamp foot 820 to the coracoid process. The detachable, spring-loaded portion may be a temporary aid tool to position the tool on the coracoid process. Unlike prior applications where a practitioner may be required to hold the plate-portion with two holes with one hand, in the present application, a practitioner may set and tighten the screws through the two holes with both hands. However, in other examples, the additional bone screw may be fixed to the coracoid process through the superior clamp foot 820 prior to the superior clamp foot 820 being separated from the body of the clamping tool 800.

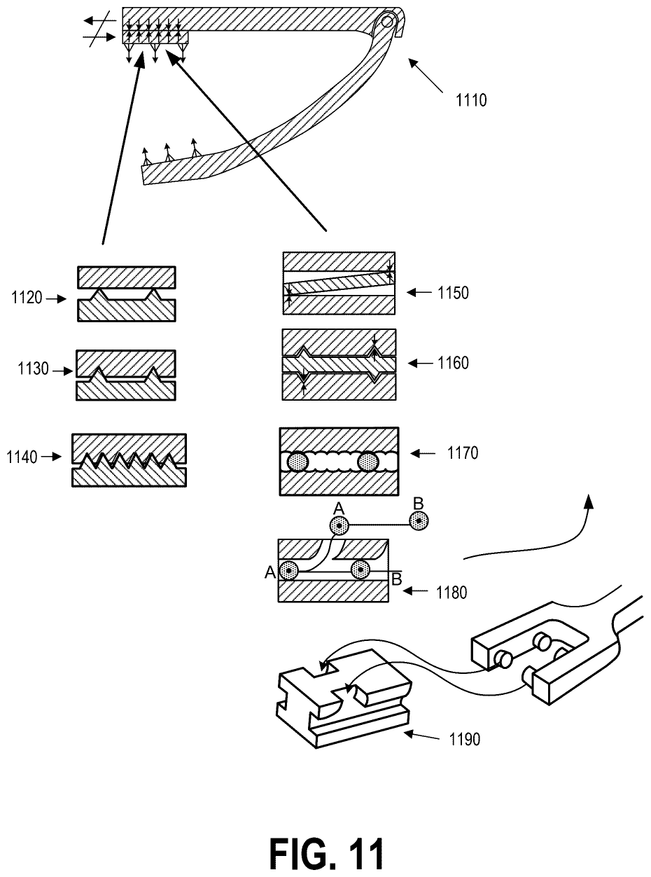

FIG. 11 illustrates examples of different interfaces that may be used to connect a superior clamp foot to a superior leg of a clamping tool, such as the removable superior clamp foot to connect to the clamp foot base. Although illustrated as a clamping tool 1110, in some examples, the interfaces may be used with the clamping tool 800 of FIG. 8.

There may be numerous options to temporarily connect the removable superior foot and the clamp foot base. In some embodiments, the removable superior foot may be connected to the clamp foot base with a certain adjustable connection force. To allow an adequately rigid fixation with the attached clamping tool, the connection interface needs to have some additional friction or geometry based locking interface, such that the friction prevents the unintended removal of the superior clamp foot from the clamping tool.

When the clamping tool is tightened about a coracoid process, a stabilizing force may act between the superior foot plane and the inferior clamp plane. In this condition, if the interface between the superior clamp foot and the clamp foot base stay planar, a slight surface roughness may cause the superior clamp foot and the clamp foot base to stay rigidly connected and may also prevent shear movement in any direction.

Example interfaces 1120, 1130 and 1140 show examples of different mating interfaces that may be used between superior clamp foot and the clamp foot base. To reduce shear movement in any direction between the superior foot plane and the inferior clamp plane, once the clamp is tightened, sawtooth like geometries (e.g., at least 2 teeth) on the superior foot plane or on the inferior clamp plane may be configured. Interface 1120 shows an example of flat superior foot plane and sawtooth like geometries on the inferior clamp plane with two sawtooth. Interface 1130 shows an example of superior foot plane with two sawtooth cavities and inferior clamp plane with two sawtooth protruded towards the superior foot plane. Interface 1140 shows an example where the superior foot plane and inferior clamp has multiple sawtooth.

Interface 1150, 1160, 1170 and 1180 show examples of different mating interfaces when the removable superior foot is indirectly connected to the clamp foot base. In some embodiments, there may be a geometrical/force configuration where the contact points are on different planes of the superior clamp foot and the clamp foot base. For example, the clamp may be oblique to the arm/block. To provide a rigid fixation in this case, a fixation against a lower and a superior plane might be necessary. A fixation on multiple points/locations may be needed. To simplify the attachment/detachment of the clamp, there may be oblique or vertical openings where pins can glide in as shown in interface 1190.

In the interface 1150, the lower plane and the superior plane may be connected via a middle plane, where an end of the lower plane is connected to an end of the middle plane and an end of the superior plane is connected to the opposite end of the middle plane. In in interface 1160, the lower plane and the superior plane may be connected via a middle plane, where both surfaces of the middle plane include sawtooth geometry protruded outwards, and both the lower plane and the superior plane have sawtooth like geometry that are etched inwards. In the interface 1160, the superior plane may be in contact with the middle plane where the sawtooth geometry of the superior plane is fitted with the sawtooth geometry on one surface of the middle plane. The lower plane may be in contact with the middle plane where the sawtooth geometry of the lower plane is fitted with the sawtooth geometry on the other surface of the middle plane. The superior plane may also be connected to the lower plane via pins in the middle as shown in the interface 1170.

In the interface 1190, the removable superior foot may include oblique or vertical openings. The clamp foot base may include pins. The clamp foot base may connect to the removable foot by gliding pins into oblique or vertical openings. The perspective 1180 may be a cross sectional view of the interface 1190 illustrating the gliding mechanism.

Additionally, temporary spring elements (e.g., elastic elements) may be attached (e.g. leaf springs, helical springs, rubber springs) to enable a temporary connection of the two parts under gravity, so the two parts stay connected unless higher forces (e.g., to actively disconnect) are applied.

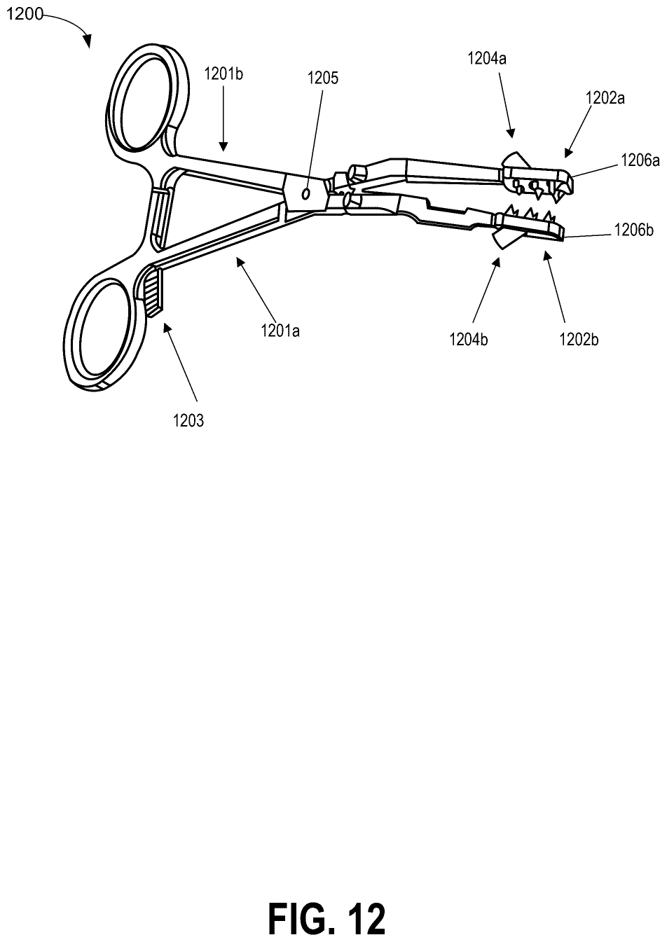

FIG. 12 illustrates an example of a scissor-like clamping tool 1200. The clamping tool 1200 may attach to a coracoid process of a scapula (e.g., the coracoid process 210 of the scapula 200). The clamping tool 1200 may include two sections, section 1201a and section 1201b, that are configured to articulate about a pivot point 1205 (e.g., a pivot screw). Each of the sections 1201a, 1201b may include a surface configured to rest on and bear against a surface of the bone. For instance, the section 1201a may include a surface 1202a of a first clamp foot 1206a (e.g., a superior clamp foot) and the section 1201b may include a surface 1202b of a second clamp foot 1206b (e.g., an inferior clamp foot). As the sections 1201a, 1201b articulate about the pivot point 1205, the surface 1202a may be considered to be an opposable surface with respect to the surface 1202b. The inner surfaces of the first and second clamp feet 1206a, 1206b may be textured (e.g., to improve a grip on a bone, such as the coracoid process). For instance, as shown in FIG. 12, the first and second clamp fect 1206a, 1206b may include “teeth”, though other textures are possible.

In some examples, the clamping tool 1200 may include one or more components 1203 that are configured to lock the sections 1201 of the clamping tool 1200 in place. The components 1203 may include any components that restrain movement of the sections 1201 in order to maintain pressure of the surfaces 1202 upon a bone. As shown in the examples of FIG. 12, the components 1203 may include a rachet locking mechanism.

The surfaces 1202a, 1202b of the first and second clamp feet 1206a, 1206b may include holes. For instance, the surface 1202a may include a hole 1204a and the surface 1202b may include a hole 1204b (collectively, “holes 1204”). The holes 1204 may be configured to receive one or more bone screws to tighten the clamping tool 1200 onto the bone. The one or more bone screws may be configured to stabilize the clamping tool 1200 (e.g., to further reduce relative movement between the clamping tool 1200 and the scapula). The holes 1204 may be guided and at an angle about the surfaces 1202. End of the one or more bone screws may be embedded in bone (e.g., either in a hole separately drilled or the one or more bone screws may be impacted into an unprepared bone) and opposite ends of the one or more bone crews may be directly attached to the clamping tool 1200 and at the other end of the surface.

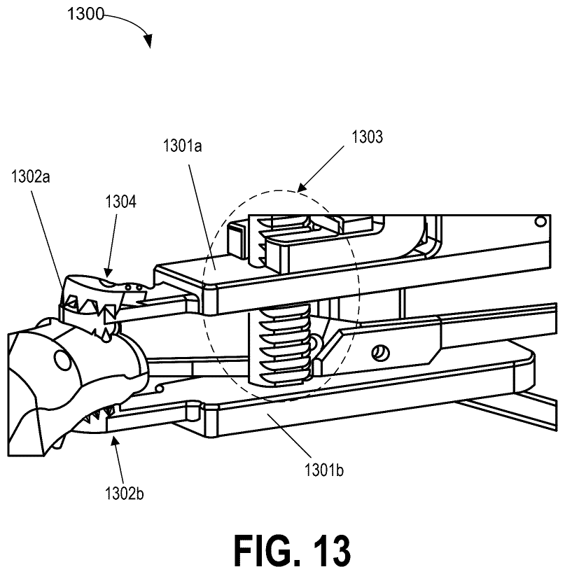

FIG. 13 illustrates an example of a clamping tool 1300 with a rachet locking mechanism 1303. The clamping tool 1300 may include two sections, section 1301a and section 1301b (collectively, “section 1301”). Each of the sections 1301 may include a clamp foot configured to rest on and bear against a surface of a bone. For instance, the section 1301a may include a superior clamp foot 1302a and the section 1301b may include an inferior clamp foot 1302b.

The clamp foot 1302a may be an opposable foot with respect to the clamp foot 1302b. One or both of the clamp feet 1302a and/or 1302b (collectively, “clamp feet 1302”) may be textured (e.g., to improve a grip on the bone). For instance, as shown in FIG. 13, the clamp feet 1302 may include “teeth”, though other textures are possible.

Clamping tool 1300 may include one or more components 1303 that are configured to lock the sections 1301 of the clamping tool 1300 in place. The components 1303 may include any components that restrain the movement of the sections 1301 in order to maintain pressure of the clamp feet 1302 upon the bone. As shown in the examples of FIG. 13, the components 1303 may include a rachet locking mechanism.

One or both of the superior and inferior clamp feet 1302a, 1302b may include holes. For instance, the superior clamp foot 1302a may include a hole 1304. The hole 1304 may be configured to receive a bone screw to tighten the clamping tool 1300 onto the bone. The bone screw may be configured to stabilize the clamping tool 1300 (e.g., to further reduce relative movement between the clamping tool 1300 and the scapula). The holes 1304 may be guided and at an angle about the surfaces of the superior clamp foot 1302a. The bone screw may be embedded in bone (e.g., either in a hole separately drilled or the bone screw may be impacted into an unprepared bone) and opposite ends of the one or more bone crews may be directly attached to the clamping tool 1300 and at the other end of the superior clamp foot 1302a.

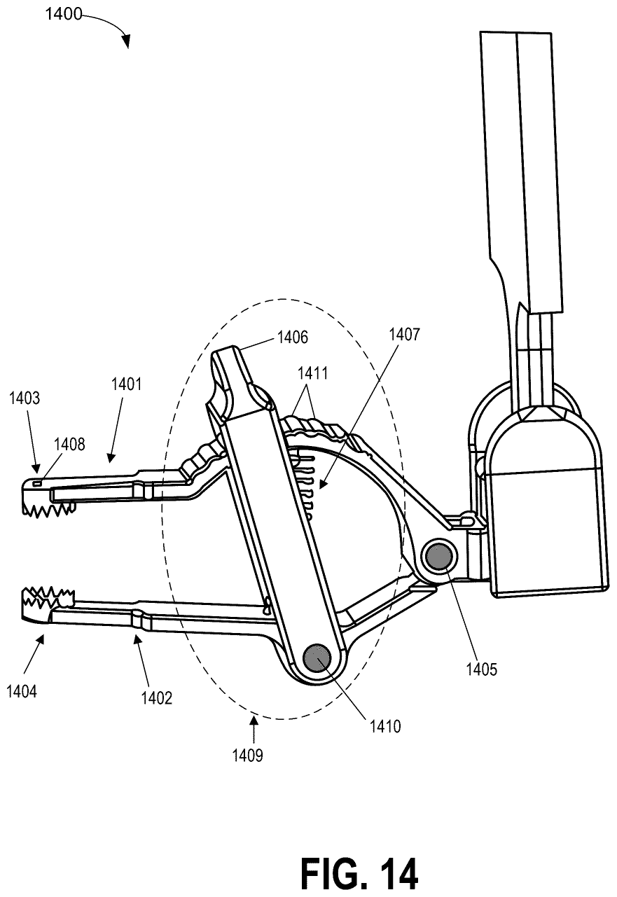

FIG. 14 illustrates an example of a clamping tool 1400 with a rachet locking mechanism 1406 and a preloaded spring 1407. The clamping tool 1400 may include two legs, a superior leg 1401 and an inferior leg 1402. The superior and inferior legs 1401 and 1402 may include clamp feet 1403 and 1404 configured to rest on and bear against a surface of the bone. For instance, the superior leg 1401 may include the superior clamp foot 1403 and the inferior leg 1402 may include the clamp foot 1404. The superior leg 1401 and the inferior leg 1402 may be connected through a hinge 1405.

The superior clamp foot 1403 may be an opposable foot with respect to the inferior clamp foot 1404. One or both of the clamp fect 1403 and/or 1404 may be textured (e.g., to improve a grip on the bone). For instance, as shown in FIG. 14, the superior clamp feet 1403 or the inferior clamp foot 1404 may include “teeth”, though other textures are possible.

The clamping tool 1400 may include a locking handle 1406 that is configured to lock the superior leg 1401 and the inferior leg 1402 of the clamping tool 1400 in place. The locking handle 1406 may include any components that restrain movement of the superior leg 1401 and the inferior leg 1402 in order to maintain pressure of the superior clamp foot 1403 and the inferior clamp foot 1404 upon a bone. As shown in FIG. 14, the locking handle 1406 may include a rachet locking mechanism 1409.

Part of the superior leg 1401 may be arched outwards away from the inferior leg 1402. The exterior part of the arch may be textured with one or more ridges 1411 to facilitate the rachet locking mechanism 1409. The locking handle 1406 may define a cavity that allows for the superior leg 1401 to pass through the cavity of the locking handle 1406. An end of the locking handle 1406 may be fixed on the inferior leg 1402. An opposite end of the locking handle 1406 may be free to move along the arch.

The clamping tool 1400 may include a preloaded spring 1407. An end of the preloaded spring 1407 may be connected to the center of the arch and the opposite end of the preloaded spring 1407 may be connected to the inferior leg 1402. The preloaded spring 1407 may provide a biasing force that pushes the ridges 1411 against an inner surface of the locking handle 1406 to prevent the superior leg 1401 and the inferior leg 1402 from moving apart from one another. The practitioner may move the locking handle 1406 toward the superior clamp foot 1403 and the inferior clamp foot 1404 along the ridges 1411 to close the clamping tool 1400 about a bone, such as a coracoid process. The practitioner may move the locking handle 1406 away from the superior clamp foot 1403 and the inferior clamp foot 1404 along the ridges 1411 to open the clamping tool 1400 and allow the clamping tool 1400 to separate from the bone.

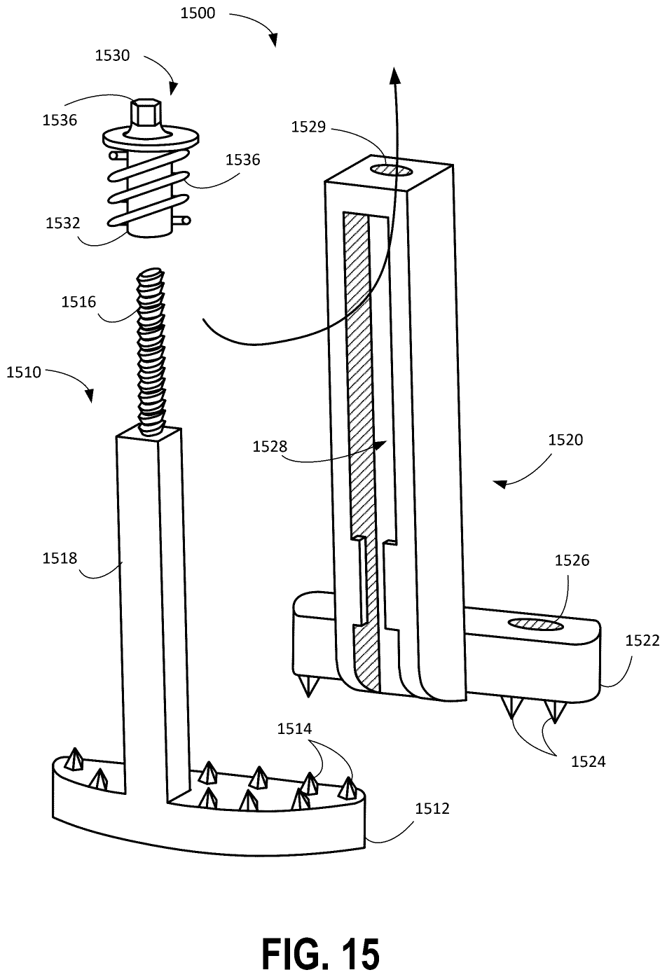

FIG. 15 illustrates an example of a clamping tool 1500 with a slide fit mechanism. The clamping tool 1500 may include an inferior leg 1510, a superior leg 1520, and a locking screw 1530. The inferior leg 1510 may include an inferior clamp foot 1512 that includes a plurality of teeth 1514. The inferior leg 1510 may include a threaded upper end 1516 that is narrower than a main body portion 1518 of the inferior leg 1510.

The superior leg 1520 may include a superior clamp foot 1522 that includes a plurality of teeth 1524. The superior leg 1520 may define a cavity 1528 (e.g., a channel) that is configured to accept the main body portion 1518 and the threaded upper end 1516 of the inferior leg 1510. For example, the main body portion 1518 of the inferior leg 1510 may be configured to slide into and reside within the cavity 1528 of the superior leg 1520 when the clamping tool 1500 is in use. For instance, when the main body portion 1518 is residing within the cavity 1528 of the superior leg 1520, the threaded upper end 1516 of the inferior leg 1510 may extend out of a hole 1529 in the superior leg 1520 and the inferior clamp foot 1512 may be aligned with the superior clamp foot 1524 such that the inferior and superior clamp feet 1512, 1524 may affix to a bone, such as a coracoid process.

When the main body portion 1518 is residing within the cavity 1528 of the superior leg 1520, the locking screw 1530 may be configured to screw onto and tighten around the threaded upper end 1516 of the inferior leg 1510. For example, the locking screw 1530 may define a threaded cavity (not shown) in a body portion 1532 of the locking screw 1530 that is sized and configured to accept the threaded upper end 1516 of the inferior leg 1510. The practitioner may tighten the locking screw 1530 using a tool that grips the head 1536 of the locking screw 1530. For instance, as the locking screw 1530 is turned clockwise, the locking screw 1530 may move down the threaded upper end 1516 of the inferior leg 1510, which may cause the inferior and superior clamp feet 1512, 1524 to move closer together and tighten about the bone. Conversely, as the locking screw 1530 is turned counter-clockwise, the locking screw 1530 may move up the threaded upper end 1516 of the inferior leg 1510, which may cause the inferior and superior clamp fect 1512, 1524 to move farther apart to loosen the clamping tool 1500 from the bone. Further, when the clamping tool 1500 is secured to the bone, a bone screw (e.g., the bone screw 302) may extend through a hole 1526 in the superior clamp foot 1520 to affix the superior clamp foot 1520 to the coracoid process. In addition to the screw, the locking screw 1530 may tighten and secure the inferior and superior clamp feet 1512, 1524 about the coracoid process.

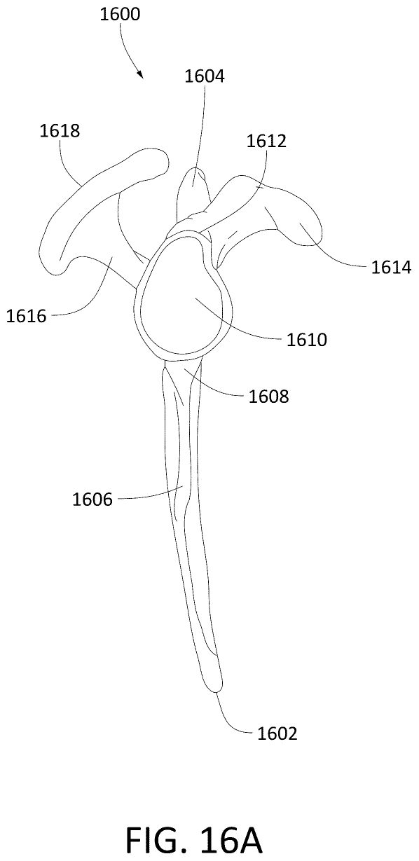

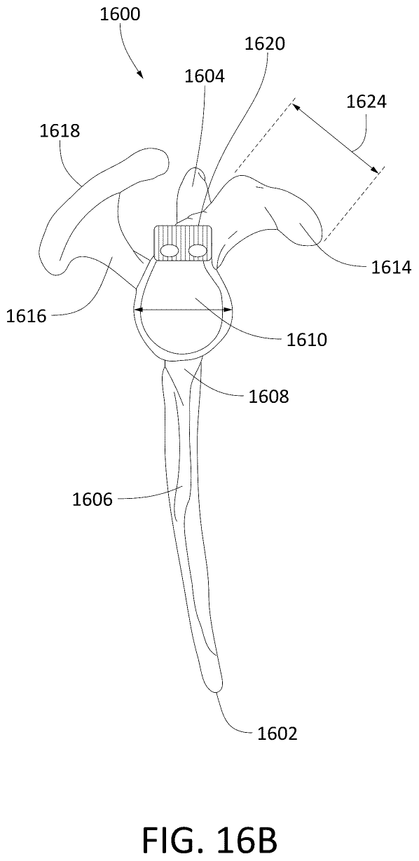

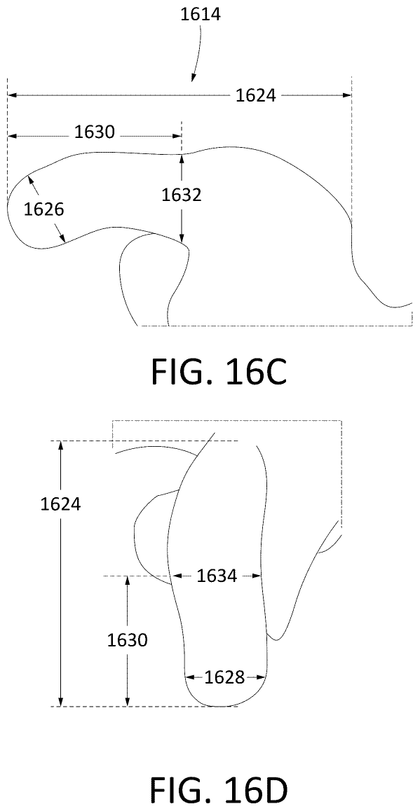

FIGS. 16A-B show examples of a scapula 1600. Example locations for placing a navigational tracker are shown. The scapula 1600 may include an inferior angle 1602. The inferior angle 1602 may be where medial and lateral borders of the scapula 1600 meet. The scapula 1600 may include a superior angle 1604, for example opposite the inferior angle 1602. The superior angle 1604 may be where superior and medial borders of the scapula 1600 meet. The scapula 1600 may include a lateral angle 1606, for example between the superior angle 1604 and the inferior angle 1602. The lateral angle 1606 may be where superior and lateral borders of the scapula 1600 meet.

The scapula 1600 may include an infraglenoid tubercle 1608. The infraglenoid tubercle 1608 may be a projection of the scapula 1600. Adjacent the infraglenoid tubercle 1608 for example, may be a glenoid cavity 1610. The glenoid cavity 1610 may extend from the infraglenoid tubercle 1608 to a supraglenoid tubercle 1612. The glenoid cavity 1610 may be where the head of the humerus is disposed to form a joint of the scapula 1600. The infraglenoid tubercle 1608 may be a projection of the scapula 1600 adjacent to the glenoid cavity 1610 and opposite the infraglenoid tubercle 1608.

The scapula 1600 may include a coracoid process 1614. The coracoid process 1614 may include a hook-shaped bone structure, for example projecting anterolaterally from a superior aspect of a scapular neck. The coracoid process 1614 may be useful for placement of a navigational tracker. The scapula 1600 may include a spinous process 1616. The spinous process 1616 may include a ridge on a posterior surface of the scapula 1600. An acromion 1618 may include a ridge, for example attached to the spinous process 1616. The acromion 1618 may articulate with the clavicle at an acromioclavicular joint.

FIGS. 16A-D illustrate example scapulas. FIG. 16A illustrates a scapula 1600 with a navigational tracker 1620 placed thereon. For example, the navigational tracker 1620 may be attached to the scapula 1600 with a fastener (e.g., as herein). The navigational tracker 1620 may be attached to the scapula 1600 using a bone screw and/or a clamping tool (e.g., clamping tool 300, 300b, 300c, 300d, and/or preloaded clamp 500, and/or clamping tool 800, 910, 940, 970, 1000, 1200, 1300, 1400, 1500).

The navigational tracker 1620 may be used before and/or during a surgical procedure. The navigational tracker 1620 may be used to track a location (e.g., movement) of a clamping tool (e.g., clamping tool 300, 300b, 300c, 300d, and/or preloaded clamp 500, and/or clamping tool 800, 910, 940, 970, 1000, 1200, 1300, 1400, 1500), for example during drilling and/or reaming of a bone (e.g., humeral bone and/or scapula). Additionally, or alternatively, the navigational tracker 1620 may be used to track a location of a clamping tool and/or an implant (e.g., as herein), for example while placing the implant. The navigational tracker 1620 may be used to track a location (e.g., movement) of a surgical instrument (e.g., drill) and/or a drill guide (e.g., surgical guide), for example during drilling and/or reaming (e.g., of a bone). For example, the navigational tracker 1620 may be used to track a location of a clamping tool, surgical instrument, and/or drill guide relative to the patient (e.g., bone).

The navigational tracker 1620 may include an optical sensor/camera, electromagnetic sensor, a navigation array (e.g., navigation array 840, 842), and/or a mini tracker sensor. While the navigational tracker 1620 is shown placed at the supraglenoid tubercle 1612 in FIG. 16B, the navigational tracker 1620 may be placed at alternative or additional positions. For example, a navigational tracker 1620 may be placed at the coracoid process 1614 and/or the glenoid cavity 1610. The navigational tracker 1620 may be placed at an upper portion (e.g. near a supraglenoid tubercle) of the glenoid cavity 1610 proximate the supraglenoid tubercle 1612 for example.

FIGS. 16C and 16D are examples of a coracoid process 1614. The coracoid process 1614 may include a coracoid length 1624, a coracoid tip height 1626, a coracoid tip width 1628, a coracoid distance from the coracoid tip to a midpoint 1630, a coracoid midpoint height 1632, and/or a coracoid midpoint width. The coracoid length 1624, coracoid tip height 1626, coracoid tip width 1628, coracoid distance from the coracoid tip to a midpoint 1630, coracoid midpoint height 1632, and/or coracoid midpoint width may be measured before and/or during surgery, for example for planning a surgical procedure.

The coracoid length 1624 may range between 29.6 mm and 49.51 mm. A preferred range for the coracoid length 1624 may include a mean of 41.60 millimeters (mm) with a standard deviation of 4.04 mm. The coracoid tip height 1626 may range between 5.62 mm and 14.26 mm. A preferred range for the coracoid tip height 1626 may include a mean of 9.05 mm with a standard deviation of 1.83 mm. The coracoid tip width 1628 may range between 8.46 mm and 19.65 mm. A preferred range for the coracoid tip width 1628 may include a mean of 13.09 mm with a standard deviation of 2.06 mm. The coracoid distance from the coracoid tip to a midpoint 1630 may range between 14.80 mm and 24.75 mm. A preferred range for the coracoid distance from the coracoid tip to a midpoint 1630 may include a mean of 20.80 mm with a standard deviation of 2.02 mm. The coracoid midpoint height 1632 may range between 7.40 mm and 15.77 mm. A preferred range for the coracoid midpoint height 1632 may include a mean of 11.12 mm with a standard deviation of 1.97 mm. The coracoid midpoint width 1634 may range between 10.54 mm and 18.28 mm. A preferred range for the coracoid midpoint width 1634 may include a mean of 14.59 mm with a standard deviation of 2.07 mm. A surgical instrument, clamping tool, drill guide, and/or navigational tracker may be selected based on one or more of the (e.g., measurement(s) of) the coracoid length 1624, coracoid tip height 1626, coracoid tip width 1628, coracoid distance from the coracoid tip to a midpoint 1630, coracoid midpoint height 1632, and/or coracoid midpoint width.

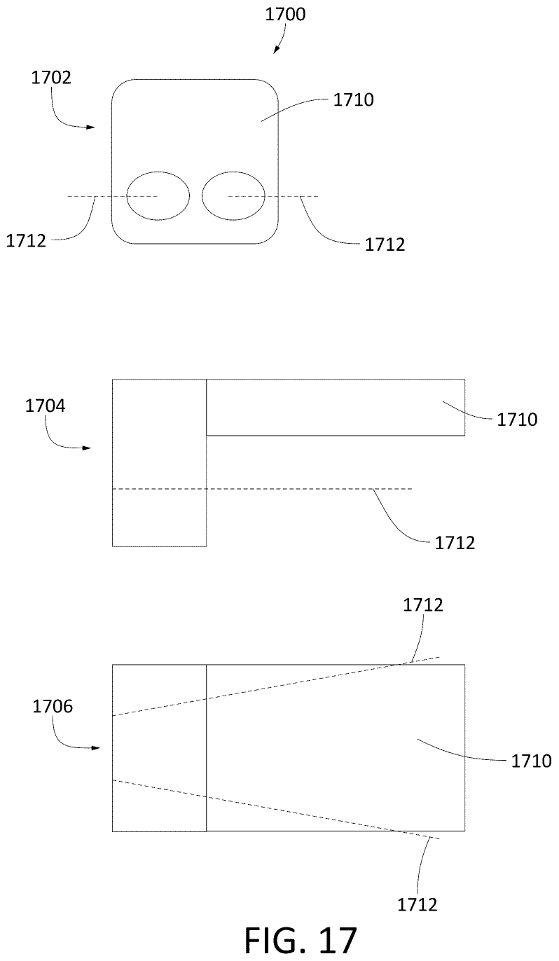

FIG. 17 illustrates example 1702, 1704, 1706 screw trajectories and drill guides 1710. For example, the screws 1712 may include bone screws. The example trajectory 1702 shows a drill guide 1710 and screws 1712. The screws drill guide 1710 may be oriented such that screws 1712 are fastened laterally into a bone. For example, screws 1712 may be fastened laterally into a glenoid cavity (e.g., 1610 as shown in FIG. 16B). The screws may be fastened into the glenoid cavity, for example near the supraglenoid tubercle.

Example trajectory 1704 shows another example of a screw 1712 fastened laterally into a bone. For example, the drill guide 1710 may rest on or near the supraglenoid tubercle. The screw 1712 may (e.g., then) be fastened into the glenoid cavity.

Example trajectory 1706 shows screws 1712 fastened into a bone at an angle to the drill guide 1710. Additionally, or alternatively, the screws 1712 may be fastened into a bone at an angle between the screws 1712. The angle may be selected based on the bone type, properties, and/or measurements.

A screw 1712, for example in example trajectory 1702, 1704, and/or 1706, may be used to fasten a navigational tracker to the bone. Additionally, or alternatively, a screw 1712 may be used to fasten an implant to the bone. A screw as herein may refer to a bone screw or a pin in some examples.

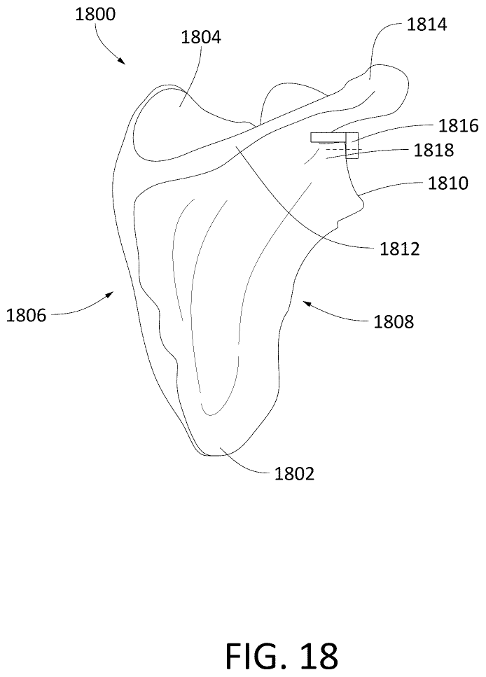

FIG. 18 illustrates another example scapula 1800. The scapula 1800 may include an inferior angle 1802. The scapula 1800 may include a superior angle 1804, for example opposite the inferior angle 1802. The scapula 1800 may include a spine 1812, for example proximate the superior angle 1804. The spine 1812 may extend to an acromion 1814. The spine may extend to a medial border 1804 opposite the acromion 1814. The medial border 1806 may extend from the spine 1812 to the inferior angle 1802.

The scapula 1800 may include a lateral border 1808. The lateral border 1808 may extend between the inferior angle 1802 and a glenoid 1810. The glenoid may include a glenoid cavity (e.g., as herein). The glenoid may be used for attachment of the navigational tracker. For example, a user may attach a navigational tracker (e.g., as herein) near a supraglenoid tubercle. The user may attach the navigational tracker using a screw 1818, for example guided by a drill guide 1816. The user may rest the drill guide 1816 on or near the supraglenoid tubercle (e.g., proximate the glenoid 1810). The user may (e.g., then) insert the screw 1818 into the glenoid 1810.

The user may use the glenoid for attaching the navigational tracker as bone quality may be sufficient for secure attachment of the navigational tracker. Positioning the navigational tracker at the glenoid may additionally or alternatively avoid exposure of tissue unnecessarily, for example where a user may otherwise attach a navigational tracker and/or a pin. As the glenoid and/or the supraglenoid tubercle are often exposed (e.g., during surgery) and have minimal tissue attachment, a user may select the glenoid and/or the supraglenoid tubercle for attachment of a navigational tracker.

The user may select the labrum of a supraglenoid tubercle for placing the navigational tracker. However, the labrum may be removed during exposure of the scapula, for example during bone surface preparation. Additionally, or alternatively, the labrum region may be close to a reaming location, for example for an ovoid anatomic glenoid implant. The navigational tracker may interfere with reaming and/or the reamer may affect tracking. The user may (e.g., therefore) decide to place the navigational tracker at the glenoid and/or the supraglenoid tubercle.

The user may select the glenoid cavity for placement of the navigational tracker, for example for a circular glenoid implant. As a user may place the implant approximate an inferior aspect of the glenoid (e.g., towards the lateral border 1808 and inferior angle 1802), the user may select the glenoid cavity (e.g., proximate the supraglenoid tubercle) for placing the navigational tracker. For example, the user may insert a screw 1818 to secure the navigational tracker to the glenoid 1810. The user may insert a screw without impacting critical regions proximate a base of the acromion 1814 and/or the coracoid (e.g., process), for example avoiding fracture risk. For example, a user may place a plurality of screws 1818 bicortically (e.g., as in 1706). Additionally, or alternatively, the user may use a clamping tool (e.g., as herein) to place the navigational tracker (e.g., at the glenoid).

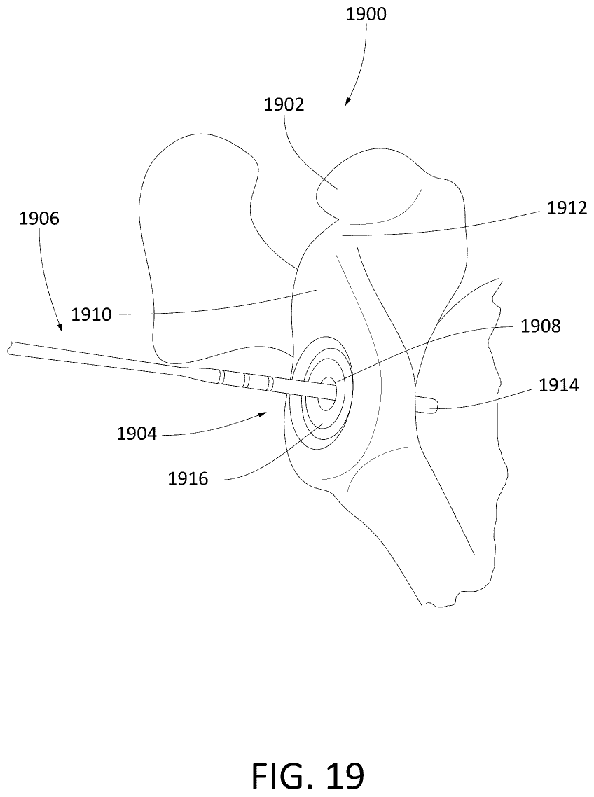

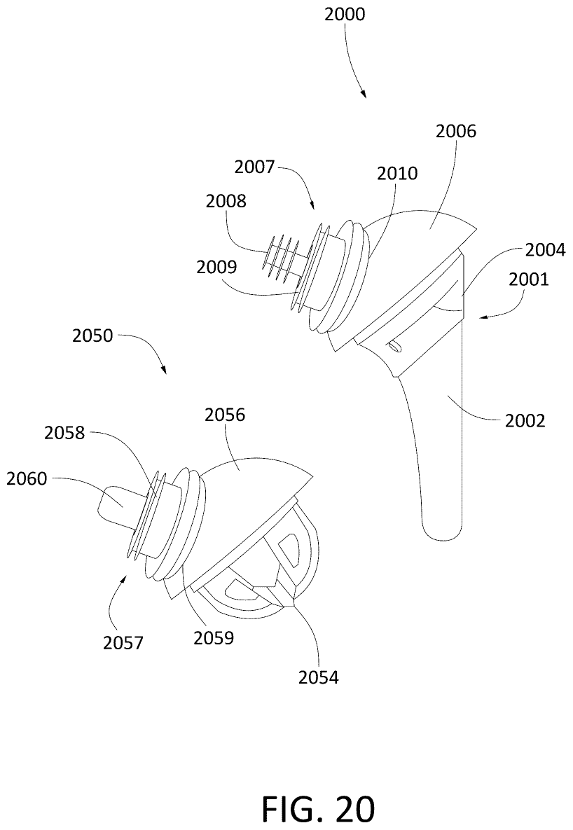

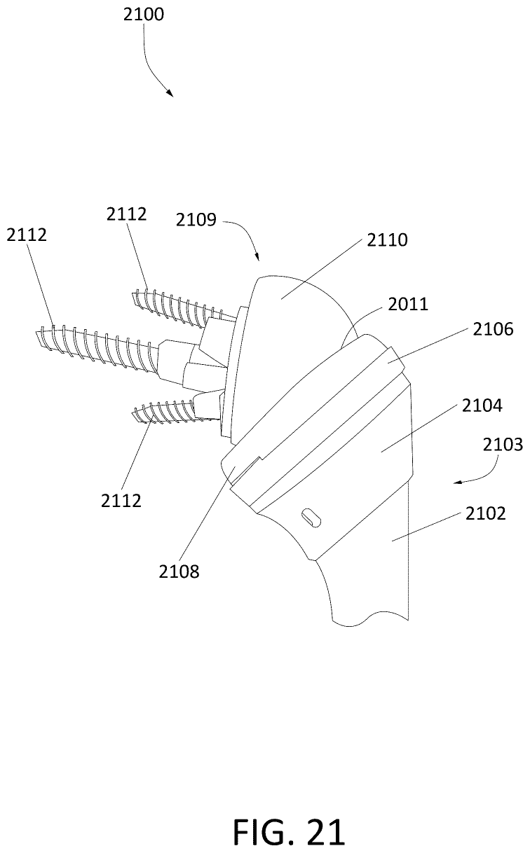

FIG. 19 illustrates an example of a drilling procedure 1900 of a glenoid 1904. The user may use a drill bit 1906 to drill a surface of the glenoid, for example using a drill guide (e.g., as herein). The user may drill a cavity 1908 in the glenoid 1904, for example for placement of an implant (e.g., as in FIG. 20 and/or FIG. 21). For example, a tip 1914 of the drill bit 1906 may extend through the glenoid 1904. Additionally, or alternatively, the user may ream near the cavity 1908 to form a ridge 1916. For example, the user may ream near the cavity to form a plurality of ridges configured to abut an implant (e.g., as in FIG. 20 and/or FIG. 21). The user may drill and ream simultaneously (e.g., using one surgical instrument). The user may (e.g., then) place the implant in the cavity 1908 and/or abutting a ridge 1916.

As discussed herein, the user may place a navigational tracker, for example before drilling and/or reaming the cavity 1908 and/or ridge 1916. The user may place the navigational tracker at the glenoid 1904, for example at a region 1910 of the glenoid 1904 proximate the supraglenoid tubercle 1912. The region 1910 of the glenoid may be sufficiently close for tracking using the navigational tracker during a surgical procedure, while being far enough to not interfere with drilling and/or reaming.

Additionally, or alternatively, the user may place a navigational tracker at the supraglenoid tubercle 1912. In some examples, the user may place a navigational tracker at the coracoid process 1902. The user may place a navigational tracker at any combination of the region 1910 of the glenoid 1904, the supraglenoid tubercle 1912, and/or the coracoid process for example. The user may place a navigational tracker using a screw (e.g., bone screw), pin, and/or a clamping tool (e.g., as herein).

FIG. 20 illustrates example implants 2000, 2050. A stemmed implant 2000 includes a humeral implant portion 2001. The humeral implant portion 2001 may be configured to be placed in a humeral bone of a patient. The humeral implant portion 2001 may include a stem 2002 and a humeral implant 2004. The user may insert the stem into a cavity in the humeral bone. Additionally, or alternatively, the user may insert the humeral implant 2004 into the humeral bone, for example near a surface of the humeral bone.

The humeral implant 2004 may be attached to a humeral head 2006. The user may insert the humeral head 2006 into the humeral implant 2004, for example during a surgical procedure. The implant 2000 may include a glenoid implant portion 2007. The glenoid implant portion 2007 may include a prong 2008 and/or a ring 2009. The user may insert the prong 2008 into a cavity of the glenoid (e.g., 1908). Additionally, or alternatively, the user may insert the ring 2009 into the glenoid, for example at a ridge (e.g., 1916). The glenoid implant portion 2007 may include a plurality of rings 2009. A (e.g., each) ring 2009 of the plurality of rings may be configured to fit a (e.g., a corresponding) ridge (e.g., 1908). The user may insert the humeral implant portion 2001 and the glenoid implant portion 2007 such that the humeral implant portion 2001 and the glenoid implant portion 2007 form a joint 2010, for example a ball and socket joint.

The stemless implant 2050 may include a humeral implant 2054. The user may insert the humeral implant 2054 into a cavity in the humeral bone. The humeral implant 2054 may be attached to a humeral head 2056. The user may insert the humeral head 2056 into the humeral implant 2054, for example during a surgical procedure.

The stemless implant 2050 may include a glenoid implant portion 2057. The glenoid implant portion 2057 may include a prong 2060 and/or a ring 2058. The user may insert the prong 2060 into a cavity of the glenoid (e.g., 1908). Additionally, or alternatively, the user may insert the ring 2058 into the glenoid, for example at a ridge (e.g., 1916). The glenoid implant portion 2057 may include a plurality of rings 2058. A (e.g., each) ring 2058 of the plurality of rings may be configured to fit a (e.g., a corresponding) ridge (e.g., 1908). The user may insert the humeral implant portion 2054 and the glenoid implant portion 2057 such that the humeral implant portion 2054 and the glenoid implant portion 2057 form a joint 2059, for example a ball and socket joint.

FIG. 21 illustrates an example reverse implant 2100. The reverse implant 2100 may include a humeral implant portion 2103 and/or a glenoid implant portion 2109. The humeral implant portion 2103 may include a stem 2102. The stem 2102 may be attached to a humeral implant 2104. The user may insert the stem 2102 and/or the humeral implant 2104 into a humeral bone of a patient. The user may insert a stemless humeral implant (e.g., the humeral implant 2104) into the humeral bone in some examples.

The humeral implant portion 2103 may be configured as a socket. For example, the humeral implant portion 2103 may include an attachment portion 2106 and/or a socket portion 2108. The attachment portion 2106 may attach the socket portion 2108 to the humeral implant 2104. The socket portion 2108 may be configured to receive a head portion 2110, for example of the glenoid implant portion 2109. The user may attach the glenoid implant portion 2109 to the glenoid, for example with bone screws 2112. For example, the user may attach the head portion 2110 to the glenoid of the patient with one or more screws 2112. The user may insert the humeral implant portion 2103 and the glenoid implant portion 2109 such that the humeral implant portion 2103 and the glenoid implant portion 2109 form a joint 2111, for example a ball and socket joint.

A user may use a navigational tracker for computer assisted placement of surgical instruments, for example in shoulder surgery. Additionally, or alternatively, the user may select a location (e.g., bone) for placement of a navigational tracker to be minimally invasive and/or not interfere with a surgical procedure. For example, a user may place the navigational tracker with less bone trauma (e.g., without drilling and/or screwing). The user may register the location of the glenoid using the navigational tracker. The user may (e.g., then) register the location of the supraglenoid tubercle, for example to avoid interference of a navigational tracker (e.g., placed at the supraglenopid tubercle) and an implant.

1. A method of attaching a navigational tracker, the method comprising:
attaching a navigational tracker to a foot of a clamping tool, the foot of the clamping tool configured to receive a screw;
attaching the clamping tool to a coracoid process;
inserting a screw into the coracoid process through the foot of the clamping tool; and
removing at least a portion of the clamping tool from the coracoid process, wherein the foot of the clamping tool remains attached to the coracoid process.

2. The method of claim 1, wherein the navigational tracker comprises at least one electromagnetic sensor.

3. The method of claim 1, comprising drilling or reaming a glenoid cavity with an instrument guided by navigational assistance using the navigational tracker.

4. The method of claim 3, comprising:
attaching an implant in the cavity of the glenoid by placing at least one screw through the implant and into the glenoid with a surgical instrument guided by navigational assistance using the navigational tracker.

5. The method of claim 4, wherein the implant comprises an ovoid glenoid implant.

6. The method of claim 4, wherein the implant comprises a circular glenoid implant.

7. The method of claim 1, wherein the foot of the clamping tool comprises a superior clamp foot, and wherein the clamping tool comprises:
an inferior leg comprising an inferior clamp foot located at a distal end of the inferior leg, wherein the inferior clamp foot is configured to contact an inferior surface of the coracoid process;
a superior leg comprising the superior clamp foot located at a distal end of the superior leg, wherein the superior clamp foot is configured to contact a superior surface of the coracoid process, and wherein the superior clamp foot comprises a hole that is configured to receive the screw; and
a clamp closing mechanism configured to tighten the superior clamp foot and the inferior clamp foot about the coracoid process, and
wherein the method comprises attaching a coupling interface attached to the navigational tracker to the superior clamp foot.

8. The method of claim 7, wherein removing the at least a portion of the clamping tool from the coracoid process comprises removing the inferior leg comprising the inferior clamp foot and the clamp closing mechanism from the coracoid process, wherein the superior clamp foot of the clamping tool and the coupling interface remain attached to the coracoid process, and wherein the navigational tracker is attached to the superior clamp foot via the coupling interface.

9. The method of claim 8, comprising attaching the coupling interface to the superior clamp foot after the clamping tool is attached to the coracoid process.

10. A method for shoulder surgery with navigation assistance, the method comprising:
attaching a navigational tracker to a clamping tool, the clamping tool comprising a foot configured to abut a surface of a scapula;
attaching the clamping tool to the scapula;
positioning the navigational tracker to guide a surgical instrument;
drilling or reaming a glenoid cavity in the scapula using the surgical instrument; and
attaching an implant in the glenoid cavity of the scapula with navigation assistance using the navigational tracker.

11. The method of claim 10, wherein the foot comprises a first foot, the clamping tool comprises the first foot and a second foot, the second foot configured to abut a surface of the scapula, and wherein attaching the clamping tool to the scapula comprises attaching the first foot and the second foot to a coracoid process of the scapula.

12. The method of claim 11, wherein attaching the implant in the glenoid cavity of the scapula comprises placing at least one screw through the implant and into the glenoid cavity using navigation assistance using the navigational tracker.

13. The method of claim 12, wherein the at least one screw comprises a first screw and a second screw, and wherein the method comprises placing the first screw and the second screw in a surface of the glenoid such that the first screw and the second screw are not parallel.

14. The method of claim 10, wherein the navigational tracker comprises at least one electromagnetic sensor.

15. A method for shoulder surgery with navigation assistance using a navigational tracker, the navigational tracker comprising a pin configured to be inserted into a scapula, the method comprising:
inserting the pin of the navigational tracker into the scapula;
positioning the navigational tracker to guide a surgical instrument;
drilling or reaming a glenoid in the scapula using the surgical instrument; and
attaching an implant in the glenoid of the scapula with navigation assistance using the navigational tracker.

16. The method of claim 15, wherein the scapula comprises a coracoid process, the coracoid process comprising a superior surface and an inferior surface, and wherein inserting the pin of the navigational tracker into the scapula comprises inserting the pin of the navigational tracker into the coracoid process laterally between the superior surface and the inferior surface of the coracoid process.

17. The method of claim 15, wherein the scapula comprises a coracoid process, the coracoid process comprising a superior surface and an inferior surface, and wherein inserting the pin of the navigational tracker into the scapula comprises inserting the pin of the navigational tracker into the superior surface of the coracoid process.

18. The method of claim 15, wherein the scapula comprises a supraglenoid tubercle, and wherein inserting the pin of the navigational tracker into the scapula comprises inserting the pin of the navigational tracker into the supraglenoid tubercle.

19. The method of claim 15, wherein the scapula comprises a glenoid, and wherein inserting the pin of the navigational tracker into the scapula comprises inserting the pin of the navigational tracker into the glenoid.

20. The method of claim 15, wherein the navigational tracker comprises at least one electromagnetic sensor.
